(19) United States
(12) Patent Application Publication
(10) Pub. No.: US 2022/0356428 A1
(43) Pub. Date: Nov. 10, 2022

(54) PANCREAS-ON-A-CHIP AND USES THEREOF

(71) Applicant: Children's Hospital Medical Center, Cincinnati, OH (US)

(72) Inventors: Anjaparavanda Naren, Cincinnati, OH (US); Kyushik Mun, Montgomery, OH (US); Kavisha Arora, Katy, TX (US)

(21) Appl. No.: 17/621,403

(22) PCT Filed: Jul. 1, 2020
(86) PCT No.: PCT/US20/40429

§ 371 Date: Dec. 21, 2021

Related U.S. Application Data
(60) Provisional application No. 62/870,140, filed on Jul. 3, 2019.

(51) Int. Cl. C12M 3/06; C12M 3/00; B01L 3/00

(52) U.S. Cl. CPC C12M 23/16; C12M 21/08; B01L 3/502707

(57) ABSTRACT

Disclosed herein are microfluidic devices that may be used to mimic human organ systems, in particular, pancreatic function, and methods of using same. In particular, disclosed are microfluidic devices that may include a first chamber having a plurality of pancreatic ductal epithelial cells (PDECs), a second chamber having a plurality of pancreatic islets, and a permeable membrane fluidly connecting the chambers. The disclosed devices and methods may be used for the study of pancreatic cell function, for the development of therapeutics, or for the development of personalized therapeutics wherein the cells of the device are obtained from an individual in need of such treatment.

### CROSS-REFERENCE TO RELATED APPLICATIONS

This application claims priority to and benefit of U.S. Provisional Application No. 62/870,140 filed Jul. 3, 2019, the contents of which are incorporated in their entirety for all purposes.

### BACKGROUND

The cystic fibrosis transmembrane conductance regulator (CFTR) protein is located on the apical membrane of epithelial cells in multiple organs, including lung, sweat gland, gastrointestinal tract, and pancreas, and its dysfunction is responsible for the clinical manifestations of cystic fibrosis (CF)¹⁻⁴. CFTR is a cyclic AMP (cAMP)-dependent chloride and bicarbonate transport channel and plays an important role in maintaining salt and water balance on the epithelial surface. Defective CFTR channel function lowers the water content in the lumen, which leads to the development of a thick and viscous mucus on the epithelial surfaces in CF-affected organs³. To date, more than 2000 CFTR mutations have been identified since the first discovery of CF in 19385. CFTR mutations are classified into six categories according to the primary molecular defect of the CFTR protein:synthesis (class I), trafficking process (II), gating (III), conductance(IV), mRNA stability (V), and CFTR stability (VI)6.

CF-related diabetes (CFRD) is a frequent and deadly complication in CF. A patient with CF has an increasing risk of developing diabetes with age of 5% per year, reaching 50% by age 40^{7,8}. CFRD affects 2% of children, 19% of adolescents, and as high as 50% of adults⁷. Glucose imbalance due to CFRD has been correlated with increased morbidity and mortality in patients with CF. This calls for a need to develop approaches to study CFRD and identify therapeutic measures to potentially manage disordered glucose metabolism in CFRD. CFRD is complex as it exhibits the features of both the lack of insulin typical of type 1 diabetes (T1D) and the insulin resistance typical of type 2 diabetes (T2D)⁹. Whether a lack of CFTR function in CF patients directly manifests into CFRD remains unclear. Patients with CFRD show more severe side effects with significant loss of lung function and imbalanced nutrition than CF patients without diabetes¹⁰. CFTR is highly expressed in the pancreatic ductal epithelial cells (PDECs)^{1,11-13}, which are located in close proximity to pancreatic islets¹⁴; however. the functional relationship between these two cell types in CFRD remains unclear.

### BRIEF SUMMARY

Disclosed herein are microfluidic devices that may be used to mimic human organ systems, in particular, pancreatic function, and methods of using same. In particular, disclosed are microfluidic devices that may include a first chamber having a plurality of pancreatic ductal epithelial cells (PDECs), a second chamber having a plurality of pancreatic islets, and a permeable membrane fluidly connecting the chambers. The disclosed devices and methods may be used for the study of pancreatic cell function, for the development of therapeutics, or for the development of personalized therapeutics wherein the cells of the device are obtained from an individual in need of such treatment.

### BRIEF DESCRIPTION OF THE DRAWINGS

This application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1. Isolation of patient-derived pancreatic ductal epithelium and islet cells. a Schematic representation of the total pancreatectomy with islet autotransplantation (TPIAT) procedure for pancreatitis patient. b Digested pancreatic remnant cell pellets were obtained following isolation of islet cells for infusion. c Hematoxylin and eosin (H&E stain) image demonstrates that the remnant cell pellet contains pancreatic islets, ductal epithelial cells, and acinar cells. d Pancreatic islets were identified by adding dithizone solution, where the color turned to red, and g isolated by manual pipetting. e Pancreatic ductal tissues were isolated by microdissection from the pancreatic remnant cell pellet following TPIAT. f H&E staining showed that pancreatic ductal epithelial cells were surrounded by collagen and connective tissue. h Pancreatic ductal epithelial cells (PDECs) were isolated from the ductal tissue and embedded in Matrigel matrix. PDECs grew into large spheres over time. i PDECs extend from the isolated organoid to form a monolayer on the surface of the substrate. j Ductal epithelial cell monolayers were re-formed into an organoid structure in the Matrigel and grew into a large sphere again. k Revived ductal epithelial cells following cryopreservation were embedded in the Matrigel and formed into large spheres over time. Scale bars: 50 μm (f), 100 μm (c, g-j and k), 500 μm (e), and 1000 μm (d)

FIGS. 2A-2L. Characterization of pancreatic ductal epithelial cells. (2A-2F, 2L: Characterization of organoids). 2A. Characterization of pancreatic ductal organoids using epithelial cell markers, 2B cytokeratin 19 (KRT 19, 2C E-cadherin, 2E sodium transport channel (ENaC), and 2F ZO-1. 2D Hematoxylin and eosin (H&E) image shows the orientation of pancreatic ductal epithelial cells in spheroid of the organoid. (2G-2K: Characterization of monolayer of pancreatic ductal epithelial cells (PDECs)). 2G Phase contrast and 2J H&E images show monolayer of PDECs formed from the organoids. Monolayer of PDECs showed positivity for tight junction 2H ZO-1, 2I F-actin and KRT 19, and 2K cystic fibrosis transmembrane conductance regulator (CFTR). 2L RNA-sequencing data was obtained from the pancreatic ductal organoids and verified the PDEC origin (n=4 sample preparation from the same patient). Data are mean±SD. Scale bars: 10 μm (2K), 20 μm (2B, 2C, 2E, 2F-2J enlarge), 50 μm (2D, 2G, 2J), and 500 μm (2A)

FIG. 3. Monitoring cystic fibrosis transmembrane conductance regulator (CFTR) channel function and endocrine function. CFTR channel function was monitored by stimulating cAMP with forskolin (FSK) using a fluid secretion assay for pancreatic ductal organoids (n>450 organoids; from 21 pancreatitis patients; data are mean±SE) and B short-circuit current measurement in polarized monolayer of ductal epithelial cells grown on a trans-well filter. CFTR channel is activated by FSK and inhibited by CFTRᵢₙ₋₁₇₂. C Phase contrast image shows cultured pancreatic islet in vitro. d Pancreatic islets were examined by immunofluorescence detection of insulin (green) and glucagon (red). E. Endocrine function was monitored by incubating pancreatic islets with different concentrations of glucose-containing media (100 and 450 mg/dL) for 1 h serially. Pancreatic islets were stimulated by high glucose (n=3 sample preparation from the same patient; data are mean±SD). Scale bars: 100 μm (2C) and 20 μm (2D). (p values from one-way analysis of variance (ANOVA) and adjust using Bonferroni factor: *<0.01, **<0.005, ****<1.0×10-20)

FIGS. 4A-4E. A unique microfluidic device. 4B A microfluidic device, single-channel chip, was designed to mimic pancreatic duct-like structure with branches and narrowing diameters. 4A) Pancreatic ductal epithelial cells (PDECs) were cultured in the chip and (4D) cystic fibrosis transmembrane conductance regulator (CFTR) function was monitored using iodide efflux assay with <10,000 PDECs (n=3). 4E Endocrine function was monitored with 15 pancreatic islets (n=3) 4C by incubating with 100 and 450 mg/dL glucose-containing media for 1 h serially. Secreted amount of insulin was measured using enzyme-linked immunosorbent assay (ELISA). Scale bars: 50 μm (4A) and 100 μm (4C). (p values from one-way analysis of variance (ANOVA) and adjust using Bonferroni factor: **<0.005; n=3 number of chips; data are mean±SD)

FIGS. 5A-5E. Pancreas-on-a-chip to study cystic fibrosis-related diabetes (CFRD). A small piece of non-treated head of pancreas was obtained and examined by 5A immunofluorescence microscopy with insulin and cystic fibrosis transmembrane conductance regulator (CFTR) and 5C hematoxylin and eosin (H&E) stain. It shows that pancreatic islets are located in close proximity to the pancreatic duct. To mimic pancreatic structure and function, 5B pancreas-on-a-chip has been developed that is comprised of two-cell culture chambers and a thin layer of porous membrane. 5D Pancreas-on-a-chip allows for the co-culture of pancreatic ductal epithelial cells (PDECs) on the top chamber with pancreatic islets in the bottom chamber. 5E Endocrine function of islet cells was monitored with stimulation or inhibition of CFTR function in PDECs on the top chamber. Applicant observed that CFTR channel function has a direct effect on the endocrine function. Secreted insulin was dramatically decreased (53.7%) by inhibition of CFTR function of PDECs. This in vitro model system, pancreas-on-a-chip, allows for the study of cell-cell interaction. Scale bars: 10 μm (5A), 50 μm (5C), and 100 μm (5D). (p values from one-way analysis of variance (ANOVA) and adjust using Bonferroni factor: *<0.05, **<0.005; number of chips: Chip A (n=3) and Chip B (n=4); data are mean±SE)

FIG. 6. Isolation of pancreatic ductal organoids. A schematic shows the isolation process of pancreatic ductal organoids from pancreatic ductal tissue. The pancreatic duct is digested to take PDECs out from the tissue and PDECs are spun down after filtering. PDECs are embedded in Matrigel and covered by organoid media containing growth factors.

FIG. 7. Formation of monolayer of PDECs from organoids. Pancreatic ductal organoids grown in Matrigel over time. When the organoids reach the surface, they start forming duct-like structures and PDECs come out from the organoids to form a monolayer.

FIGS. 8A-8B. Polarized monolayer of PDECs in pancreas-on-a-chip. Polarized monolayer of PDECs on a porous membrane in the chip was verified (8A) using immunofluorescence image with tight junction, ZO-1, and (8B) using epithelial volt-ohm meter to measure transepithelial electrical resistance (TEER). The chopstick electrodes were connected to Ag/AgCl wires for the measurement. Scale bar: 100 μm (8B).

FIG. 9. Comparison of endocrine function. Comparison of insulin secretion in islet cells from non-pancreatic disease patient and pancreatitis patient. (n=3 sample preparation from non-pancreatic disease and pancreatitis patient; Data are mean±SD)

FIGS. 10A-10B. Design of cell culture chamber. (10A) Cell culture chambers in pancreas-on-a-chip was designed using AutoCAD software. (10B) The chamber has branches with narrowing diameters. (unit: mm).

FIG. 11. Process for single-channel chip. The microfluidic device, single-channel chip, was fabricated using standard photolithography and soft lithography techniques. Initially, designed patterns are created on a silicon wafer through photolithography and cast uncured PDMS to have patterned PDMS layer. Bond the PDMS with glass substrate after treatment with oxygen plasma to seal the chamber.

FIG. 12. Expression of CFTR in pancreatic ductal epithelial cells Immunofluorescent microscopy of insulin and CFTR (white arrows) was performed on head of pancreas (a) and pancreatic remnant cell pellet followed by TPIAT (b). Scale bar: 20 μm.

FIG. 13. Process for double-channel chip. Schematic shows fabrication of pancreas-on-a-chip comprised of two cell culture chambers and a thin layer of porous membrane. Bond patterned PDMS layer for top chamber with porous membrane and alignment with the other PDMS layer for bottom chamber. Pancreas-on-a-chip allows for the co-culture of two different types of cells.

FIGS. 14A-14C. Effect of CFTR inhibitor on insulin secretion in double-channel chip. Insulin secretion was monitored from islet cells on the bottom chamber of the double-channel chip followed by incubation with CFTR inhibitor, 20 μM CFTRᵢₙₕ₋₁₇₂, applied to the top chamber only. Islet cells were stimulated with high glucose-containing media (450 mg/dL) at the end to verify that endocrine function was not impaired. The CFTR inhibitor did not show any effect on insulin secretion without pancreatic ductal epithelial cells (a; CASE I) or without pores on the membrane (b; CASE II) in the double-channel chip. Insulin secretion was attenuated upon inhibition of CFTR function in double-channel chip with pores (c; CASE III; taken from FIG. 5E). (p-values from one-way ANOVA and adjust using Bonferroni factor: *<0.05, **<0.005, ***<0.0005; n=3 sample preparation from the same patient; Data are mean±SD).

FIGS. 15A-15D. Functional examination of PDECs and islet cells obtained from pancreatitis/CF patient. a. CFTR function in PDECs (pancreatitis/CF patient) was observed using fluid secretion measurement and compared with 21 pancreatitis patients. The basal secretion in this patient was 20% lower than pancreatitis patient (n: the number of organoids; Data are mean±SE). b. Endocrine function was monitored using ELISA. Islet cells secreted insulin efficiently in response to highly concentrated glucose (450 mg/dL) (n=3 sample preparation from the same patient; Data are mean±SD). c. PDECs and islet cells were co-cultured in pancreas-on-a-chip and compared insulin secretion with pancreatitis/non-CF patient (FIG. 5e) (n=3; the number of chips; Data are mean±SD). d. Insulin secretion was dramatically decreased by inhibition of CFTR function from both patients, pancreatitis/non-CF patient (54%) and pancreatitis/CF patient (46%)(n=3; the number of chips; Data are mean±SD).(p-values from one-way ANOVA and adjust using Bonferroni factor: *<0.05, **<0.005, ****<1.0×10-5)

FIG. 16 is a schematic perspective view of an example of a microfluidic device for use as a pancreas-on-a-chip.

FIG. 17 is a schematic cross-sectional view of an upper plate of the microfluidic device of FIG. 16 taken along section line 17-17 of FIG. 16.

FIG. 18 is a schematic cross-sectional view of a lower plate of the microfluidic device of FIG. 16 taken along section line 18-18 of FIG. 16.

FIG. 19 is a schematic cross-sectional view of a porous membrane of the microfluidic device of FIG. 16 taken along section line 19-19 of FIG. 16.

FIG. 20 is a schematic cross-sectional view of the microfluidic device of FIG. 16 taken along section line 20-20 of FIG. 16.

FIG. 21 is an enlarged schematic view of the microfluid device of FIG. 20.

FIG. 22 is a schematic sectional view of a top chamber of the upper plate of FIG. 17.

FIG. 23 is a schematic sectional view of the top chamber of the upper plate of FIG. 17 having exemplary dimensions of various geometries.

FIG. 24 is a schematic sectional view of a bottom chamber of the lower plate of FIG. 18 having exemplary dimensions of various geometries.

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “effective amount” means the amount of one or more active components that achieves a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

For clarity of disclosure, the terms “upper,” “lower,” “lateral,” “transverse,” “longitudinal,” “bottom,” “top,” “right,” and “left” are relative terms to provide additional clarity to the figure descriptions provided below. The terms “upper,” “lower,” “lateral,” “transverse,” “bottom,” “top,” “right,” and “left” are thus not intended to unnecessarily limit the invention described herein.

In addition, the terms “first” and “second” are used herein to distinguish one or more portions of a device. For example, a first assembly and a second assembly may be alternatively and respectively described as a second assembly and a first assembly. The terms “first” and “second” and other numerical designations are merely exemplary of such terminology and are not intended to unnecessarily limit the invention described herein.

The instant disclosure relates to microfluidic devices and uses thereof. The microfluidic devices may be useful for a variety of different purposes, both as described herein and as would be readily understood by one of ordinary skill in the art. For example, the various embodiments of the device as described herein may be used in personalized medicines, wherein the device may be used to culture cells derived from the individual, and efficacy and/or safety of a therapeutic may be assayed using the device. The devices may be further used for tissue analysis, such as the ability to grow normal structures and cells from cells derived from an individual.

In one aspect, a microfluidic device, comprising a first surface at least partially defining a first chamber; a plurality of pancreatic ductal epithelial cells (PDECs) received within said first chamber; a second surface at least partially defining a second chamber; a plurality of pancreatic islets received within said second chamber; and a permeable membrane fluidly connecting said first and second chambers such that said plurality of PDECs are configured to communicate with said plurality of pancreatic islets to mimic in situ pancreatic cell function is disclosed. In one aspect, the PDECs and pancreatic islets may be derived from an individual. In one aspect, the PDECs and pancreatic islets may be derived from the same individual. In certain aspects, the individual may be one who has undergone a TPIAT. In certain aspects, said individual may be one having a disease selected from one or more of Acute Recurrent Pancreatitis (ARP) or chronic pancreatitis (CP), and cystic fibrosis (CF).

In one aspect, the first chamber further of the microfluidic device may include a first cell culture media positioned therein. The second chamber may further includes a second cell culture media positioned therein.

In one aspect, the first cell culture media and second cell culture media may comprise insulin.

The cell culture media may comprise the following components. For PDECs, the media may comprise Advanced DMEM/F-12 based medium containing HEPES, GlutaMAX, penicillin streptomycin, N2, B27, N-Acetylcysteine, and growth factors (Noggin, R-Spondin, and Epithermal Growth Factor). For pancreatic islets, the media may comprise Low glucose-containing based medium (DMEM; 100 mg/dL glucose) containing fatal bovine serum, penicillin streptomycin. In certain aspects, for the co-culture of PDECs and islets, the same media may be used. An exemplary media may be DMEM based, and may be used for both chambers. In one aspect, the media may be Advanced DMEM/F-12 (Advanced DMEM/F-12 contains ethanolamine, glutathione, ascorbic acid, insulin, transferrin, AlbuMAX® II lipid-rich bovine serum albumin for cell culture, and the trace elements sodium selenite, ammonium metavanadate, cupric sulfate, and manganous chloride, and is available from ThermoFisher Scientific.)

In one aspect, each of said plurality of PDECs may be in a monolayer. The monolayer may be a polarized monolayer.

In one aspect, the plurality of PDECs express a cystic fibrosis transmembrane conductance regulator (CFTR) protein. In one aspect, the plurality of islets secrete insulin.

In one aspect, the permeable membrane may comprise a plurality of openings extending between and fluidly connecting said first and second chambers. The plurality of openings may have of a width of from about 5 μm to about 25 μm, or about 10 μm. Suitable opening sizes will be readily understood by one of ordinary skill in the art, and may vary, depending on the desired operation of the membrane and porosity. The gap of between two pores (from center to center) may be, in certain aspects, about 25 μm. The thickness of the membrane may generally be less than about 10 μm.

In one aspect, the first surface may be in contact with said plurality of PDECs, wherein said second surface is in contact with said plurality of pancreatic islets, and wherein at least one of said first surface or said second surface at least partially includes a hydrophilic surface.

In one aspect, the hydrophilic surface may be selected from poly methyl methacrylate, acrylonitrile butadiene styrene copolymer, cyclic olefin copolymer, styrene ethylene butylene styrene, collagen, or combinations thereof.

In one aspect, the first surface may be in contact with said plurality of PDECs, wherein said second surface may be in contact with said plurality of pancreatic islets, and wherein at least one of said first surface or said second surface may have a sol-gel-modified PDMS or a collagen-coated-PDMS received thereon. The device itself may comprise any suitable material as would be appreciated in the art. Materials that may be used for the disclosed microfluidic device may comprise, for example, SiO2, glass, and synthetic polymers. Synthetic polymers can, for example, comprise polystyrol (PS), polycarbonate (PC), polyamide (PA), polyimide (PI), polyetheretherketone (PEEK), polyphenylenesulfide (PPSE), epoxide resin (EP), unsaturated polyester (UP), phenol resin (PF), polysiloxane, e.g. polydimethylsiloxane (PDMS), melamine resin (MF), cyanate ester (CA), polytetrafluoroethylene (PTFE) and mixtures thereof. The synthetic polymers are optically transparent and can include, for example, polystyrol (PS), polycarbonate (PC), and polysiloxane, e.g. polydimethylsiloxane (PDMS).

In one aspect, the first chamber may include a first branch channel and a second branch channel, wherein each of said first and second branch channels may extend in a common channel plane and intersect at a first predetermined angle. Pancreatic ducts may be connected to Acinar cells and deliver digestive enzymes to the duodenum. In vivo, pancreatic ducts are spread out entire pancreas as roots with branching from a main duct, where it connects to the duodenum. The wide channel of the device, thus, is considered as a main duct and each branch channel has narrowing diameters. This arrangement may aid the study of pancreatic pressure-related disorders. Pancreatic pressure can be modelled by adjusting flow rate. In one aspect, the first branch channel may further include a first pair of side edges extending in the common channel plane and may define a first width therebetween, wherein said second branch channel may further includes a second pair of side edges extending in the common channel plane and defining a second width therebetween, and wherein the second width may be narrower than the first width.

In further aspects, a method of measuring cystic fibrosis transmembrane conductance regulator (CFTR) protein function in an individual is disclosed. In this aspect, the method may comprise obtaining pancreatic ductal epithelial cells (PDECs) and pancreatic islets from said individual; culturing said PDECs and pancreatic islets in the device disclosed herein, wherein patient-derived pancreatic ductal epithelial cells (PDECs) may be co-cultured in a first chamber, and patient-derived pancreatic islet cells may be cultured in second chamber; assaying the function of said CFTRs in a pancreatic ductal monolayer; and measuring insulin secretion of said pancreatic islets.

In one aspect, the method may further comprise measuring one or more of fluid secretion from said PDECs in response to forskolin, measuring insulin secretion of said pancreatic islets in response to glucose, and combinations thereof.

In one aspect, the individual may have Cystic Fibrosis (CF)-related diabetes (CFRD).

In one aspect, the first and/or second chamber may be contacted with alcohol to determine CFTR function and/or endocrine function in response to said alcohol.

In one aspect, the method may be used to determine function of a CFTR mutation type, wherein said PDECs are known to contain said CFTR mutation type, and wherein function of one or both of said PDECs and/or pancreatic islets are correlated with said CRTR mutation type.

In one aspect, the method may further comprise contacting said first or second chamber with an agent suspected of improving glucose abnormalities; and measuring a glucose response in said pancreatic islets in response to said contact.

In one aspect, a method of assaying a potential treatment for one or more of Acute Recurrent Pancreatitis (ARP) or Chronic Pancreatitis (CP), and Cystic Fibrosis (CF), and Cystic Fibrosis (CF)-related diabetes (CFRD), is disclosed. In this aspect, the method may comprise

contacting a potential therapeutic agent with one or both of said first and said second chambers of the device disclosed herein; and

detecting a desired output. In one aspect, the desired output may be selected from one or both of fluid secretion from PDECs and insulin secretion from said pancreatic islets.

In one aspect, a method of making a pancreatic ductal epithelial cells (PDECs) monolayer is disclosed. In this aspect, the method may comprise digesting pancreatic duct tissue obtained from said individual, isolating PDECs from said digested pancreatic duct tissue, embedding said isolated PDECs in a matrix, preferably a basement membrane matrix, and incubating with media until one or both of an organoid and a monolayer is formed, preferably wherein said organoid or monolayer forms a duct-like structure. “Matrix,” as used herein, includes substances or mixtures of substances, which enhance proliferation, differentiation, function or organoid or organ formation of cells. Matrix material may be coated on surfaces or may be provided in voluminous applications to optimize cell attachment or allow three-dimensional cultures. Matrix usable in the context of the present invention can take a variety of shapes comprising, e.g. hydrogels, foams, fabrics or non-woven fabrics. The matrix material may comprise naturally occurring matrix substances like extracellular matrix proteins, for example, collagens, laminins, elastin, vitronectin, fibronectin, small matricellular proteins, small integrin-binding glycoproteins, growth factors or proteoglycans or may include artificial matrix substances like non degradable polymers such as polyamid fibres, methylcellulose, agarose or alginate geles or degradable polymers, e.g. polylactid. The term “matrix” may include basement membrane matrix more commonly known in the trade as “Matrigel.®”

In one aspect, the matrix may be disrupted mechanically, in the absence of trypsin, prior to said incubation with media to form said monolayer. In general, in order to obtain monolayer of epithelial cells from organoids in 3-dimensional matrix (e.g., Matrigel), Matrigel is broken down by pipetting up and down using 1 mL trypsin EDTA following wash out cell culture media with cold PBS. After 10 min incubation with the EDTA, organoids may be transferred with EDTA to 15 mL tube, followed by pipetting up and down to separate organoids into single cells. Cell culture media containing FBS may be added, followed by spinning down to obtain a cell pellet. The cells may then be resuspended with fresh cell culture media and plated on a flat surface or trans-well membrane. In one aspect, the disclosed methods may be carried out in the absence of trypsin. The matrix (Matrigel) may be fragmented by pipetting (mechanical disruption) using growth media in the culture maintaining the organoids. The organoids, matrix (Matrigel), and media may then be transferred into a 1.5 mL tube, followed by pipetting to separate the organoids from matrix. Following centrifugation at 14000 rpm for about 3 min three layers can be observed: organoids, matrix, and media (from bottom to top of the tube). The media and matrix may then be discarded, and organoids resuspended with fresh media and plated on a flat surface or trans-well membrane. A monolayer of PDECs can be obtained, keeping tight-junctions. In one aspect, the monolayer may be a polarized monolayer.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific embodiments that are disclosed and still obtain a like or similar result without departing from the spirit and scope of the invention.

### Example

Cystic fibrosis (CF) is a genetic disorder caused by defective CF Transmembrane Conductance Regulator (CFTR) function. Insulin producing pancreatic islets are located in close proximity to the pancreatic duct and impaired cell-cell signaling between pancreatic ductal epithelial cells (PDECs) and islet cells may be causative in CF. Disclosed herein, in one aspect, is an in vitro co-culturing system, termed a “pancreas-on-a-chip.” Further disclosed are methods for the microdissection of patient-derived human pancreatic ducts from pancreatic remnant cell pellets, followed by the isolation of PDECs. Applicant has found that defective CFTR function in PDECs directly reduced insulin secretion in islet cells significantly. The disclosed pancreatic function monitoring tool may be useful for, inter alia, the study of CF-related disorders in vitro, as a system to monitor cell-cell functional interaction of PDECs and pancreatic islets, characterize appropriate therapeutic measures and further the understanding of pancreatic function.

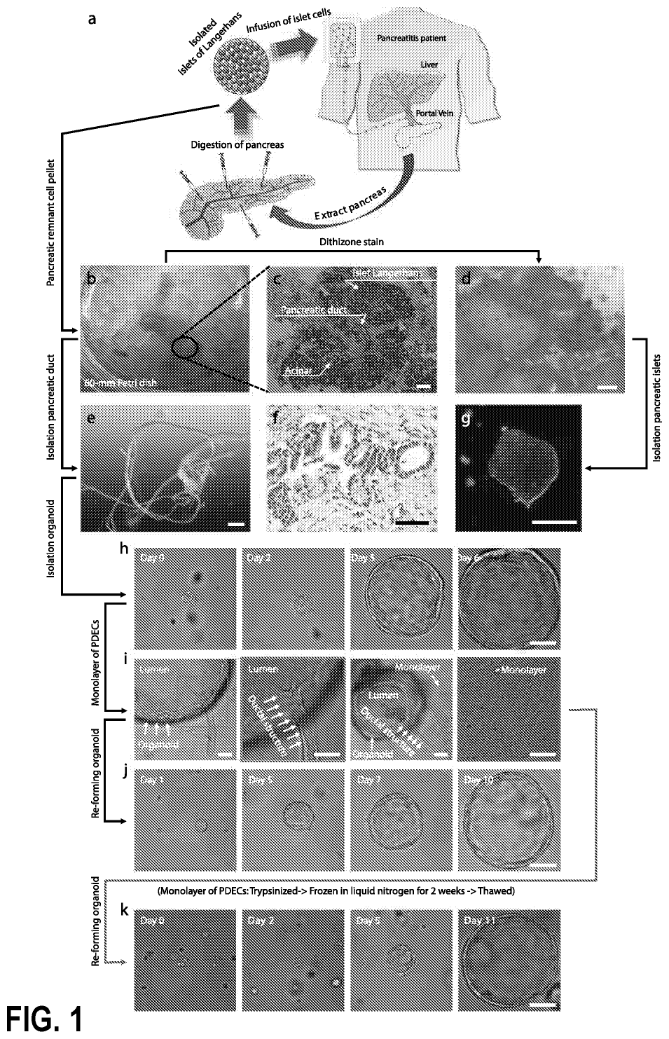

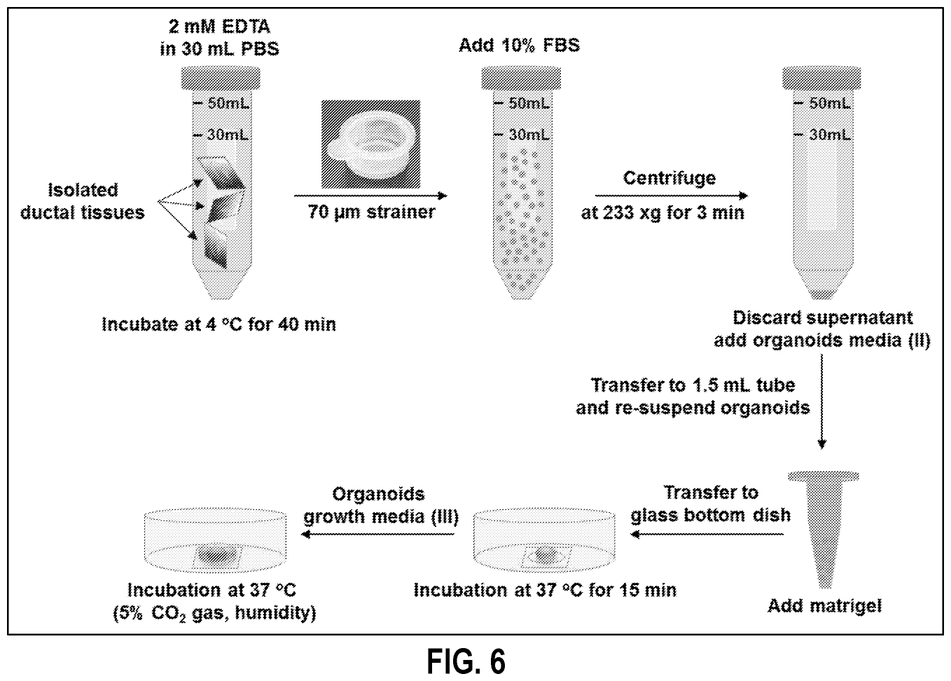

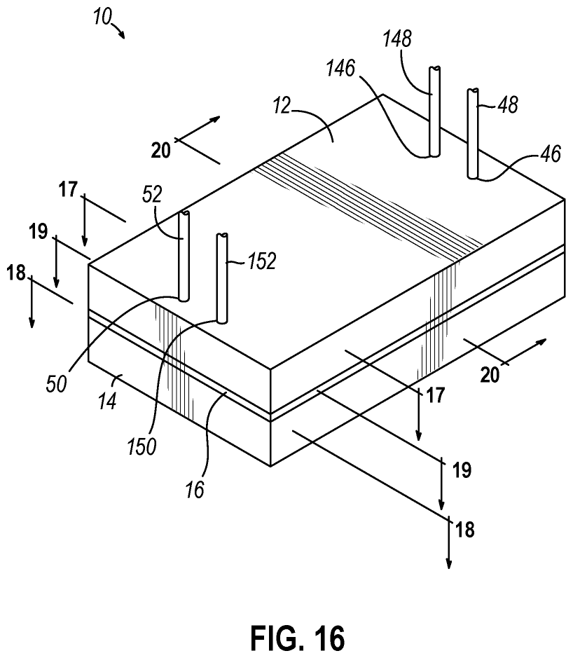

To investigate the role of CFTR in CFRD and functional correlation between PDECs and islet cells, Applicant isolated PDECs and pancreatic islets from pancreatitis patients, who underwent total pancreatectomy with islet autotransplantation (TPIAT)¹⁵. Applicant cultured these cell types in a microfluidic device, such as a microfluid device (10) (see FIGS. 6-24) to develop a “pancreas-on-a-chip,” an in vitro model system to mimic the functional interface between PDECs and pancreatic islets. The pancreas-on-a-chip allows monitoring cell-cell functional interaction directly and efficiently using only a small number of patient-derived cells. While the following description of FIGS. 1-15 may refer to various features of the pancreas-on-a-chip and the microfluidic chip, which may be used herein interchangeably, such features, particularly structural features, are more particularly described with respect to the microfluidic device (10) (see FIGS. 16-24) discussed below in greater detail. To this end, descriptions provided herein with respect to FIGS. 1-15, including, but not limited to, chambers, branches, membranes, and holes, similarly apply to these like features described with respect to microfluidic device (10) (see FIGS. 16-24) for manufacture and use thereof.

Prior to development of the pancreas-on-a-chip described herein in more detail, early versions of the microfluidic device (not shown) more generally were developed in 1979 as a miniature gas chromatograph¹⁶ and it has been exponentially innovated in functionality and design. In recent decades, microfluidics have been used as an in vitro model system for cell culture¹⁷⁻¹⁹, because of its high reproducibility, ability to mimic function and structure of organs, and some unique applications such as real-time PCR²⁰, single-cell western blot²¹, wearable sensor²², and organ-on-a-chip^{23,24}. Pancreas-on-a-chip, such as represented in one example by the microfluidic device (10) (see FIGS. 16-24), may be used to facilitate elucidating the mechanism of cross-talk between PDEC and islet cells, which is useful to understanding the relationship between CF and diabetes. Classified mutations in the CFTR gene may show variable pathological processes in the development of CFRD. Here, Applicant has successfully co-cultured patient-derived PDECs and islet cells in the same chip and observed that attenuating CFTR function in PDECs reduces insulin secretion in islet cells by 54%. This pancreas-on-a-chip is an innovative approach of developing personalized medicine to address heterogeneity in CFRDs.

Results

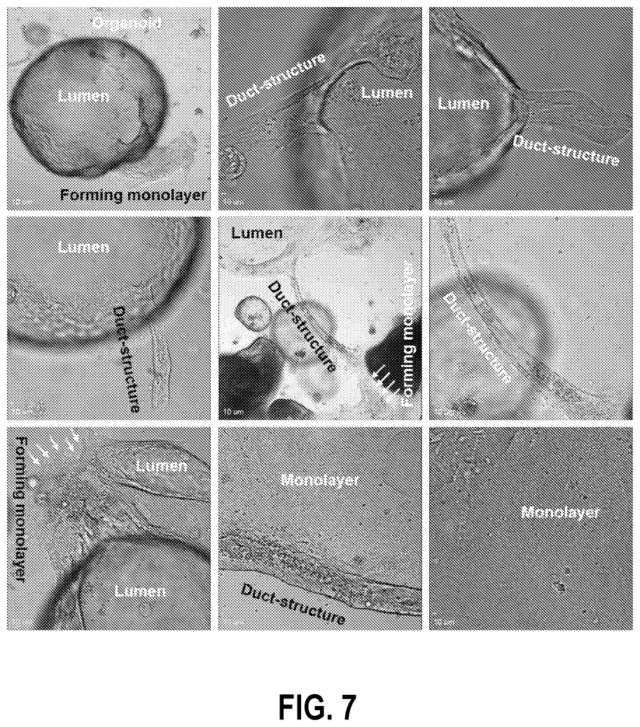

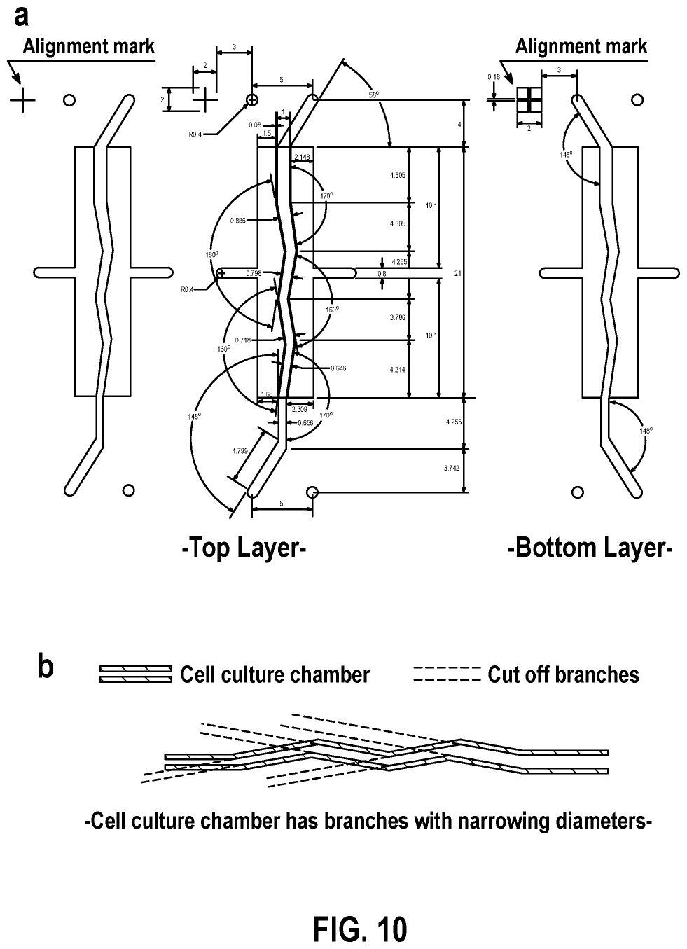

Isolation of patient-derived PDECs and pancreatic islets. Pancreatitis patients who have a debilitating course of acute recurrent or chronic pancreatitis may undergo TPIAT to relieve their pain and incapacitation, as shown in FIG. 1a. During TPIAT, the pancreas is resected and digested to isolate pancreatic islets. The islets are infused into the liver through the portal vein to engraft within the hepatic sinusoids and maintain their endocrine function. The pancreatic remnant cell pellet was obtained after islet cell isolation (FIG. 1b) and shown to contain pancreatic islets, acinar cells, and PDECs) (FIG. 1c). Pancreatic islets in the pancreatic remnant cell pellet were visualized by adding dithizone solution, which turns the color of islets to red (FIG. 1d)²⁵. The clustered red-colored pancreatic islets were manually isolated and cultured in vitro (FIG. 1g). The diameter of the isolated pancreatic islets ranged from 50 to 300 μm. From the pancreatic remnant cell pellet, Applicant successfully isolated the pancreatic duct with diameter ranging from 90 to 1000 μm by microdissection under a stereo microscope (FIG. 1e). Hematoxylin and eosin (H&E) staining of the pancreatic duct demonstrated that the predominant cells, PDECs, were surrounded by collagen and connective tissue (FIG. 10. Pancreatic ducts were enzymatically digested to separate the cells (FIG. 6). Isolated pancreatic ductal cells were subsequently embedded in Matrigel and clustered into small spherical structures after isolation (day 0) and formed organoid structures at day 1, with a luminal fluid-filled area in the center (FIG. 1h). The organoids grew into larger spheres with a diameter of approximately 400 μm at day 6 from isolation. Growth of organoids could exceed 3 mm in diameter. Organoids in Matrigel formed duct-like structures when they contacted the surface of the substrate and migrated out forming a monolayer (FIG. 1i). Although the mechanism is unclear, Applicant have consistently observed this phenomenon (FIG. 7). To assist forming a monolayer of PDECs, the organoids were hand-picked manually and transferred to a fresh culture dish or trans-well membrane following breaking down of the Matrigel. During hand-picking, the organoids were separated from the Matrigel and collapsed by pipetting. Collapsed organoids attached to the surface, and PDECs started migrating out from organoids forming a monolayer. From the monolayer of PDECs, single cells were harvested and embedded into fresh Matrigel. The PDECs re-formed organoid structures and grew over time (FIG. 1J). Furthermore, Applicant have succeeded in freezing and reviving patient-derived PDECs using transformation of the organoid-monolayer structure (FIG. 1K).

Hence, disclosed herein are methods for isolating, culturing, and expanding patient-derived ductal epithelial cells and pancreatic islets, which may be used, for example, to provide a platform for the development of personalized medicine in pancreas-related disorders such as CFRD.

Characterization of PDECs.

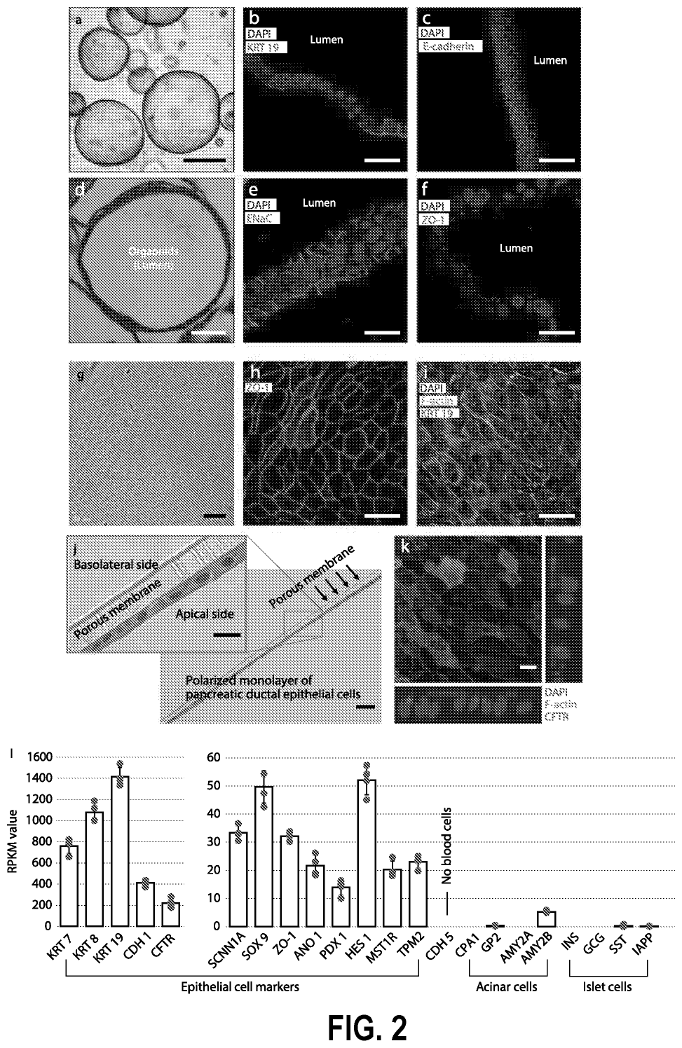

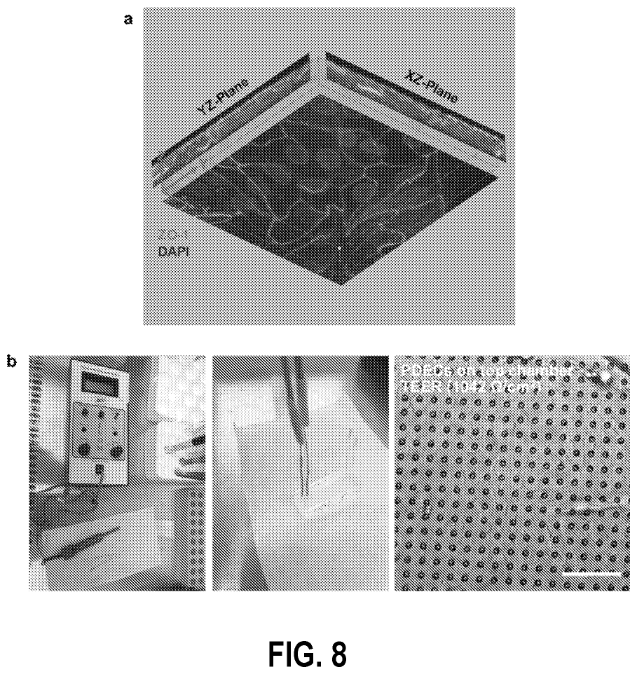

PDECs are one of the most abundant cell type present in the pancreas. Applicant intended to confirm whether the isolated cells from the pancreatic remnant cell pellet were indeed PDECs. Isolated pancreatic ductal organoids (FIG. 2A) were first examined using standard morphological H&E staining (FIG. 2D). The images demonstrated that ductal epithelial cells are located at the edge of the organoid, with a central luminal area. Immunofluorescence images showed pancreatic ductal organoids expressing epithelial cell biomarkers, cytokeratin 19 (KRT 19) (FIG. 2B), E-cadherin (FIG. 2C), sodium transport channel (ENaC) (FIG. 2E), and tight junction protein (ZO-1) (FIG. 2F). Applicant cultured PDEC-derived monolayer (FIG. 2G) from the ductal organoids and detected positive immunofluorescent signals corresponding to epithelial cell biomarkers, ZO-1 (FIG. 2H), F-actin, and KRT 19 (FIG. 2I). Paraffin-sectioned H&E images demonstrated a monolayer of PDECs (FIG. 2J) obtained from organoids. From the PDECs on the trans-well membrane, Applicant observed CFTR located on the apical membrane of the ductal epithelial cells (FIG. 2K). Polarized monolayer of PDECs on a porous membrane in a pancreas-on-a-chip was examined with immunofluorescence image of ZO-1 and measurement of transepithelial electrical resistance using epithelial volt-ohm meter (FIG. 8). Applicant performed RNA-sequencing in these organoids and verified that the organoids were of human PDECs in origin (FIG. 2L). Epithelial cell markers, cytokeratin family proteins (KRT7, KRT 8, and KRT 19), CFTR, and E-cadherin were highly expressed in the organoids; while blood cell marker (CDH 5), pancreatic acinar cell biomarkers, CPA1, GP2, and amylase (AMY2A), and pancreatic endocrine markers, insulin, glucagon, and somatostatin were not expressed. Hence, Applicant validated that the cell population processed from the pancreatic remnant cell pellet is ductal epithelium in origin, which may be used to investigate the functional coupling of two specific cell types, PDECs and pancreatic islets.

Functional measurements in PDECs and pancreatic islets.

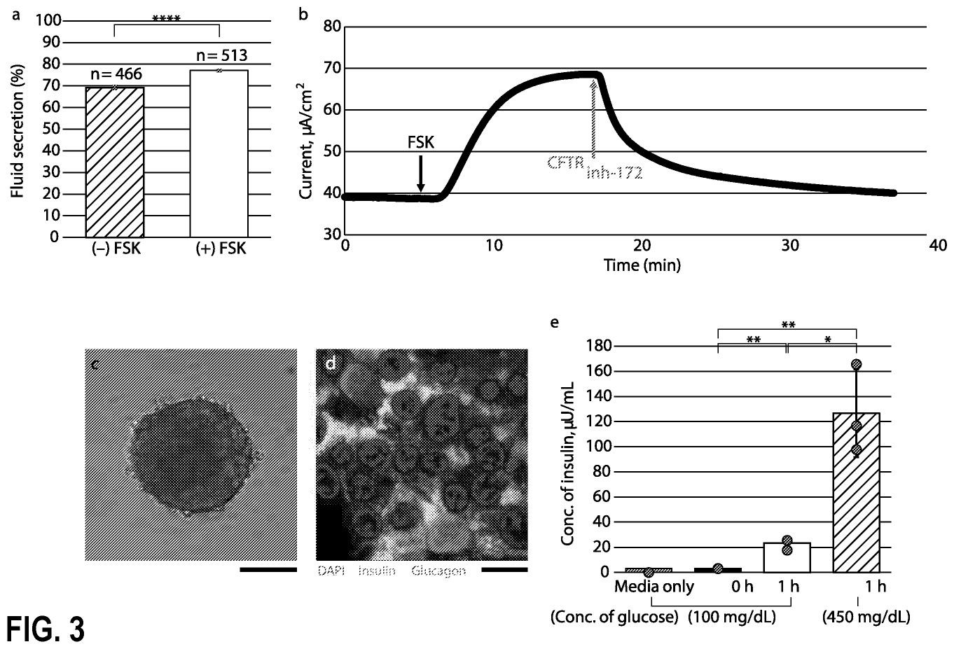

PDECs are reported to have the highest expression of CFTR in the body^{1,11-13}. CFTR function in the pancreas has a critical role in maintaining fluid and pH within the pancreatic duct to deliver digestive enzymes secreted by acinar cells into the duodenum that are important for digestive function in the intestine. CFTR function was monitored in pancreatic ductal organoids in response to the cAMP-activating agonist forskolin (FSK; 10 μM). In this assay, CFTR function was reported as a measure of fluid secretion calculated by the ratio of luminal volume to that of the entire organoid²⁶. During treatment with FSK, CFTR channels open, and chloride ions are pumped into the lumen creating an osmotic driving force for water to follow. Thus, fluid secretion is increased resulting in expansion of luminal volume. Fluid secretion was compared before and after treatment with FSK for 2 h, as shown in FIG. 3, A. Basal secretion of the ductal organoids was 60% before treatment and increased to 75% upon incubation with FSK. The graph was obtained using over 450 organoids derived from 21 pancreatitis patients. Alongside, Applicant cultured ductal organoid-derived PDECs on a transwell membrane and monitored CFTR function using short-circuit current (Isc) measurement (FIG. 3, B). The trans-electrical resistance was 1200 Ω/cm2 (1.3×10⁵ of cells). By activating the CFTR channel with FSK, electrogenic movement of chloride ions from the apical side of these cells generated Isc peak (ΔIsc=30 μA/cm2) (FIG. 3, B). The addition of CFTR inhibitor, CFTRᵢₙₕ₋₁₇₂ (20 μM), caused dramatic decrease in the Isc (FIG. 3, B).

Endocrine Function In Vitro.

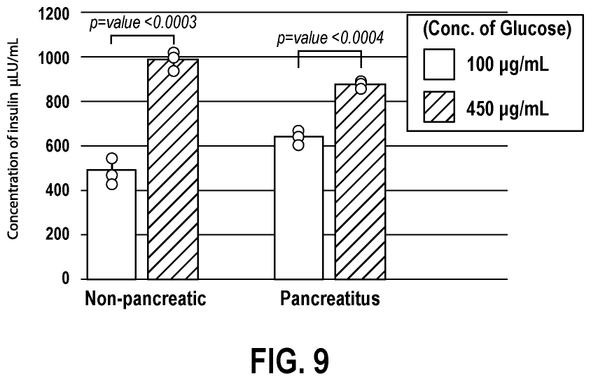

Pancreatic islets were isolated from the pancreatic remnant cell pellet of the same patient source as for PDECs and cultured in vitro using the methodology as described above (FIG. 3, C). Pancreatic endocrine function consists of production of insulin &ells) and glucagon (αcells) from pancreatic islets to maintain an appropriate blood glucose level. The pancreatic islets were examined by immunofluorescence staining specific to insulin and glucagon (FIG. 3, D). Applicant observed arrangement of (kens and αcells in the pancreatic islet marked by insulin (green) and glucagon (red), respectively (FIG. 3d). αCells are located at the edge of the clustered pancreatic islets, while (kens were distributed uniformly across the islet (FIG. 3, D). Applicant successfully monitored endocrine function in the pancreatic islets by measuring the concentration of insulin in the culture media in response to variable concentrations of glucose, 100 mg/dL (equivalent to normoglycemia) and 450 mg/dL (equivalent to hyperglycemia) (FIG. 3, E). Applicant observed that pancreatic islets secreted significantly more insulin when exposed to high glucose conditions; from 23.4 μLU/mL insulin in low glucose-containing media upon incubation for 1 h (3.3 μL U/mL at time=0 h) to 127 μLU/mL insulin in high-glucose-containing media during another 1 h incubation (FIG. 3, E). In order to verify that endocrine function in islet cells obtained from pancreatitis patient is not impaired, Applicant compared insulin secretion response to highly concentrated glucose (450 mg/dL) from non-pancreatic patient and pancreatitis patient (FIG. 9). Increased insulin secretion upon exposure to high-glucose-containing media was observed in both the patients. Thus, the overall function in islet cells was not impaired in pancreatitis patient. Therefore, Applicant generated robust in vitro functional systems to monitor CFTR function from PDECs and endocrine function from pancreatic islets, a non-limiting use of which may include the study of CFRD.

Microfluidic Device.

Using human tissue has its limitations, including limited availability and a very low viable cellular yield. The short-circuit current (Isc) assay is the gold-standard method to monitor CFTR function in real time; however, it requires approximately 1.3×10⁵ cells, and takes approximately 2 weeks to achieve a fully covered-polarized monolayer of epithelial cells on the trans-well membrane (33 mm²).

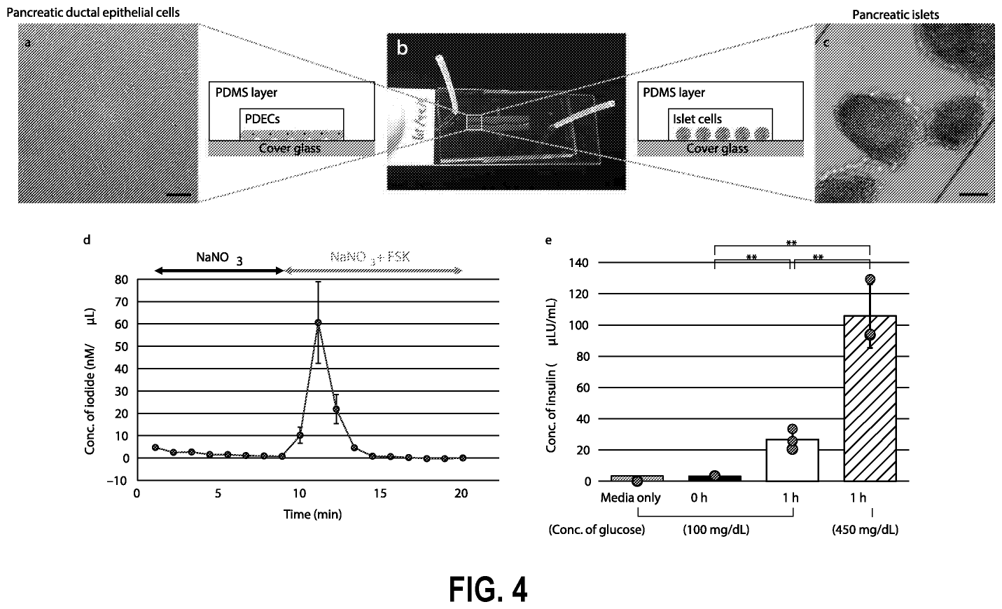

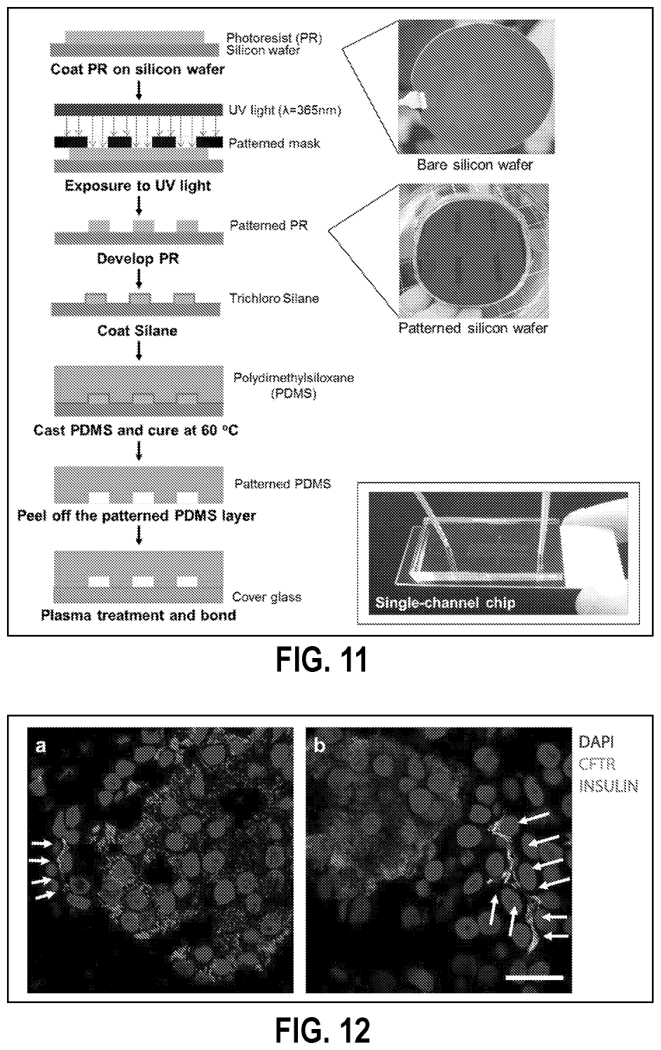

Here, Applicant developed a highly sensitive microfluidic device to monitor CFTR function from PDECs and insulin secretion from pancreatic islets cultured on the chip as shown in FIG. 4. The device, a single-channel chip (FIG. 4B), was designed to mimic pancreatic duct-like structure, which has branches with narrowing diameters (FIG. 10). The chip was fabricated using standard photolithography and soft lithography, having dimensions 26.87 mm² (area), 0.14 mm (thickness), and 3.76 mm³ (volume) for cell culture (FIG. 11). Applicant cultured PDECs (FIG. 4A) and pancreatic islets (FIG. 4C) to monitor CFTR function and insulin secretion, respectively. Total amount of cell culture media needed in the chip may be as little as 56 μL, which includes 3.76 μL for the cell culture chamber and 52 μL for two side tubings. This is in contrast to the required 200 μL (apical side) and 500 μL (basolateral side) for an Ussing chamber. The disclosed methods may be used to successfully monitor CFTR function in PDECs with <10,000 cells using the iodide efflux assay 3 days after seeding cells (FIG. 4D). In the first step of iodide efflux, cells were loaded with the iodide. Upon CFTR activation using FSK at timepoint 10 min following a baseline, the iodide was pumped out of the cells through CFTR channel, which gave an iodide peak (60±18 nM/μL). Using the single-channel chip, insulin secretion in pancreatic islets can be detected (FIG. 4E). Pancreatic islets can be cultured on the chip with 100 mg/dL glucose-containing media (basal medium). Applicant obtained 3 μLU/mL insulin from 15 pancreatic islets in the basal medium wash with no incubation. The concentration of secreted insulin increased from 27 μLU/mL (1 h in the basal medium) to 106 μLU/mL (1 h in the high-glucose-containing medium) (FIG. 4E). In this manner, Applicant developed a highly sensitive microfluidic device to measure CFTR function in PDECs and insulin secretion in islets with small numbers of cells.

Pancreas-On-a-Chip to Study CF-Related Disorders.

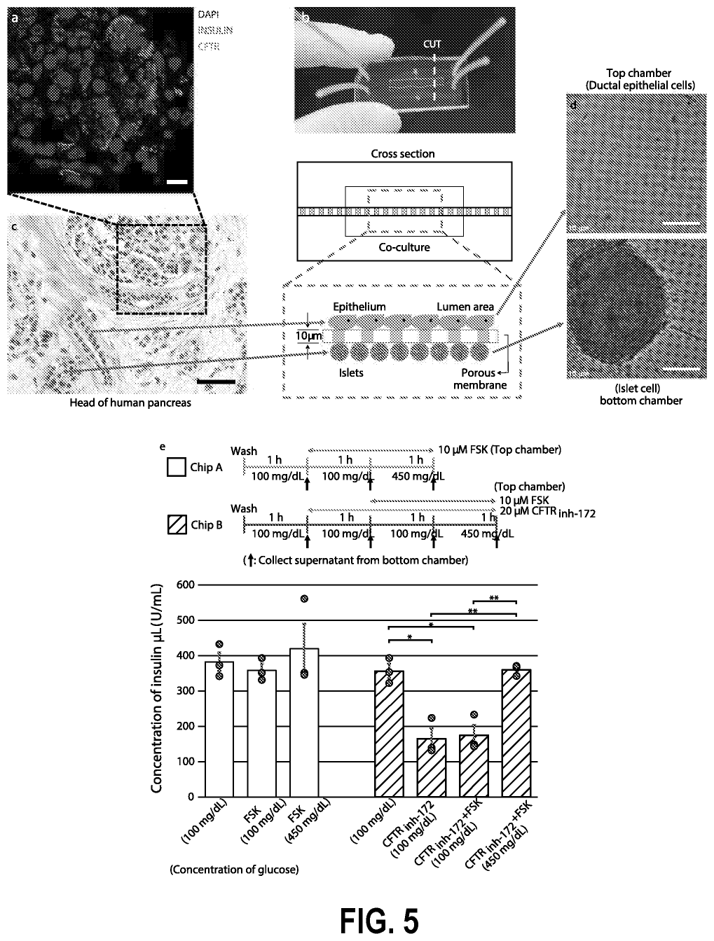

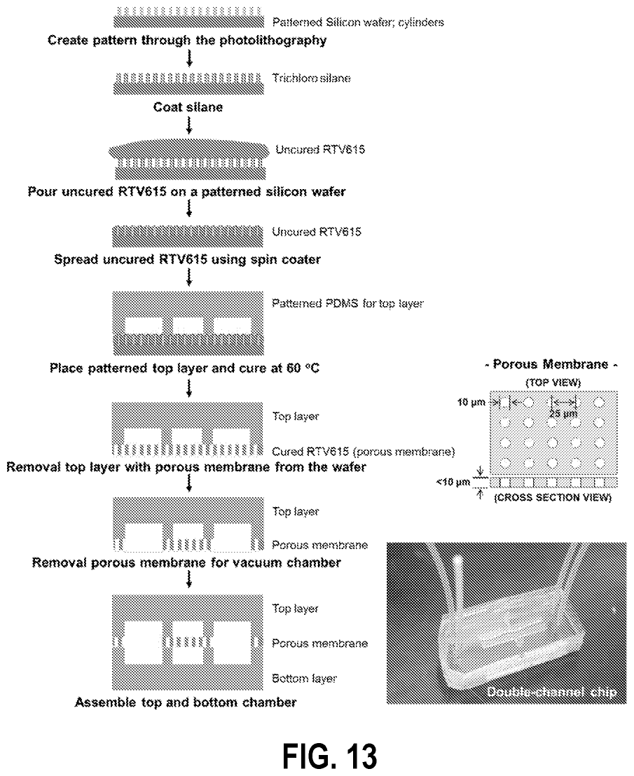

Applicant could detect that there is an interface between the ductal cells and islets based on H&E staining performed in a small piece (1 cm²) of non-treated tissue isolated from the head of the pancreas of a TPIAT patient (FIG. 5C) and immunostaining data in the same region, which was obtained from serial sections of the same sample, that showed CFTR-expressing ductal cells located in close proximity to insulin-expressing islets (FIG. 5A). Importantly, CFTR is only expressed in the PDECs, not in the pancreatic islets¹² (FIG. 12). Given the cellular proximity between ductal cells and islets, Applicant hypothesized that there is a functional coupling between these two cell types. To test this possibility, Applicant developed a pancreas-on-a-chip involving co-culturing of ductal epithelial cells and islets in two-cell culture chambers separated by a thin layer of porous membrane (<10 μm thickness) (FIG. 5B and FIG. 13). The PDECs were cultured on the top chamber and pancreatic islets were seeded in the bottom chamber (FIG. 5D).

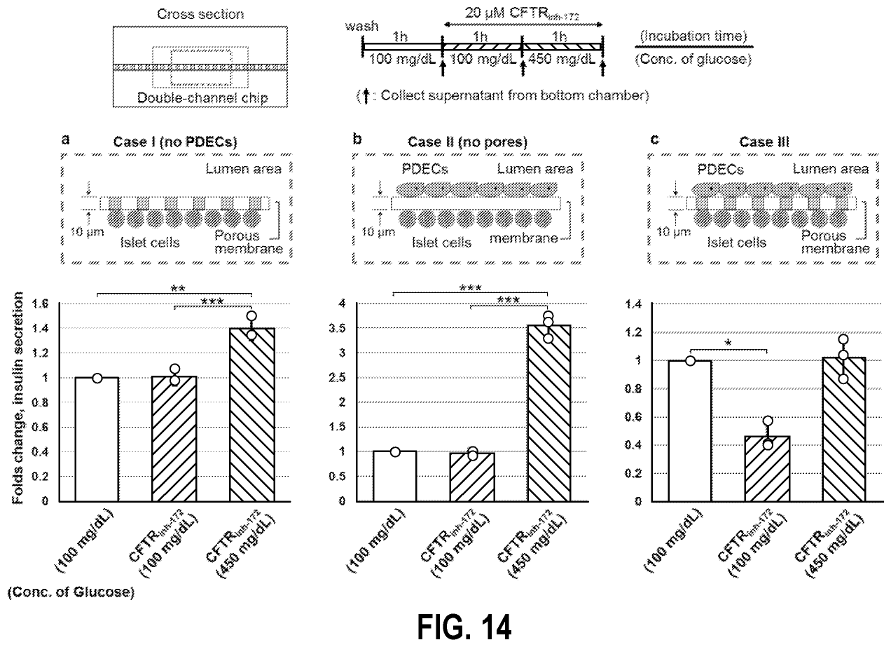

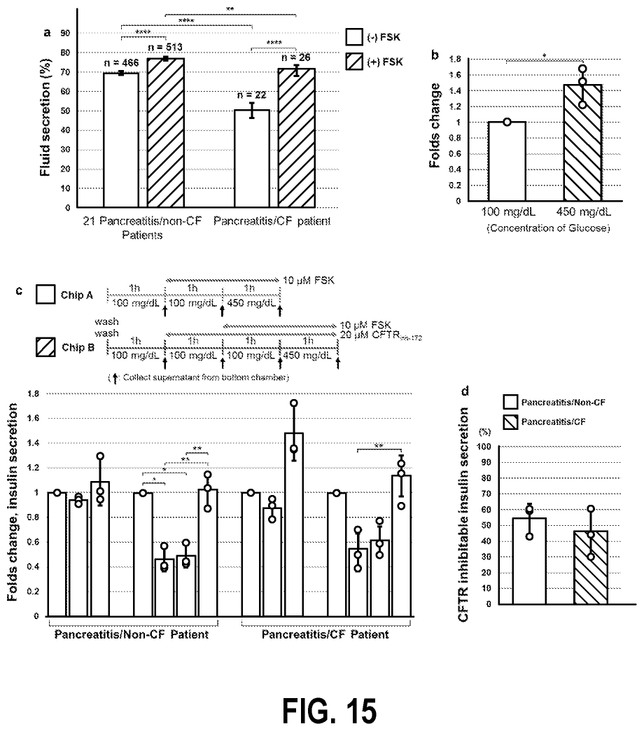

Next, Applicant tested how CFTR function may affect insulin secretion from the islet cells in this double-channel chip system. Applicant measured secreted insulin in 1 h increments from pancreatic islets in the bottom chamber following stimulation or inhibition of CFTR channel function (FIG. 5E). Stimulation of CFTR channels in PDECs in the top chamber did not show significant changes. On the other hand, upon inhibition of CFTR channel function in PDECs using CFTRᵢₙₕ₋₁₇₂ (Chip B; 20 μM, 1 h), insulin secretion was significantly decreased (53.7%; Δ191.4 μLU/mL). Applicant examined if the CFTR inhibitor directly affects insulin secretion from the islet cells (FIG. 14). Applicant cultured islet cells in the double-channel chip without PDECs (FIG. 14A) or co-cultured with PDECs lacking pores on the membrane to perturb communication between PDECs and islet cells (FIG. 14B) and added 20 μM CFTR inn-172 to the top chamber. Applicant observed that inhibition of CFTR under these conditions did not influence insulin secretion. Insulin secretion from the islet cells was not altered in the presence of FSK (10 μM) and CFTRᵢₙₕ₋₁₇₂ (20 μM) (FIG. 15). FSK stimulation did not significantly alter insulin secretion from the islet cells maintained in the basal medium (100 mg/dL glucose). At the end of the experiment, islet cells were directly exposed to high-glucose-containing media (450 mg/dL glucose; Chip A and Chip B) to verify its responsiveness to the glucose challenge, suggesting that the endocrine function was not impaired. Insulin secretion increased in both chips (Chip A:Δ60.7 μLU/mL and Chip B:Δ181.4 μLU/mL) and the amount of insulin secreted was higher than before stimulation or inhibition of CFTR function in PDECs. To further consolidate the possibility that CFTR function directly affects insulin secretion, Applicant examined the cell-cell functional correlation between PDECs and islet cells derived from pancreatitis/CF patient, who was diagnosed with very mild CF and underwent TPIAT (Table 1). The patient has ΔF508 (allele 1), R117H (allele 2), and heterozygote for SPINK1 mutation. This patient was diagnosed to have mild CF and has some CFTR function as demonstrated by the mild phenotype (i.e., body mass index: 19.84; sweat chloride: 51 mmol/L; forced expiratory volume in 1 s predicted: 114% and is not diabetic). Additionally, Applicant monitored CFTR function using fluid secretion assay and endocrine function using enzyme-linked immunosorbent assay (ELISA) as described earlier prior to co-culture of the two cell types in pancreas-on-a-chip. Applicant observed that the pancreatic ductal organoids showed partially impaired CFTR function (20% lower than non-CF pancreatitis patient in basal secretion and under 5.3% in FSK-stimulated secretion). Islet cells secreted insulin in response to the glucose challenge (FIG. 15A, 15B). Applicant co-cultured PDECs and islet cells in pancreas-on-a-chip and measured insulin secretion from the islet cells as described earlier. Applicant observed similar trend that inhibition of CFTR function affected endocrine function. Insulin secretion was decreased in pancreatitis/CF patient-derived pancreas-on-a-chip by 7.9%, but it was not significant (FIG. 15C, 15D). Overall, using this unique pancreas-on-a-chip device, Applicant demonstrated that ductal cells and islets are functionally coupled, a first-of-a-kind observation that CFTR plays a role in directly regulating insulin secretion. This observation is directly relevant to CFRD in which there is a loss of CFTR function.

TABLE 1
TPIAT Patient Summary

| | Mutations | | | Age | | FEV1 | |
|---|---|---|---|---|---|---|---|
| Patients | Allele 1 | Allele 2 | Gender | (years) | Sweat | (%) | BMI |
| Patient 1 | SPINK1 | None | F | 14 | ND | ND | 34 |
| | (pN34S) | | | | | | |
| Patient 2 | CFTR | None | M | 8 | 2 | ND | 17 |
| | (Δ508) | | | | | | |
| Patient 3 | None | None | M | 13 | ND | ND | 23 |
| Patient 4 | PRSS1 (R122H) | None | F | 9 | 16 | ND | 18 |
| Patient 5 | CFTR | None | F | 4 | 21 | ND | 15 |
| | (1454G > C Het) | | | | | | |
| Patient 6 | CFTR (R170H) | SPINK1 | M | 18 | 19 | ND | 21 |
| | | (pN34S) | | | | | |
| Patient 7 | None | None | F | 13 | 46 | 108 | 25 |
| Patient 8 | CPA1 | None | F | 13 | ND | ND | 28 |
| Patient 9 | CFTR (Δ508), | CFTR | F | 15 | 51 | 114 | 20 |
| | SPINK1 | (R117H) | | | | | |

Discussion

Applicant has successfully isolated patient-derived pancreatic ductal organoids following TPIAT and has generated a freezing and reviving protocol for pancreatic ductal epithelial cells. Pancreatic ductal organoids demonstrated growth into large spheres over time. The organoids cultured in 3D matrix allows for the efficient harvest of pure pancreatic ductal epithelial cells among multiple cell types that are present in the pancreatic remnant cell pellet. The organoids can be grown effectively from a limited number of cells to form a functional unit. The 3D organoid formation with luminal area internally has been observed in other organs, including lung²⁷, liver²⁸, and intestine²⁹. This is a repeated observation of duct-like formation from the pancreatic ductal organoids. This ductal formation may further be used to elucidate mechanisms involved in the development of the pancreatic duct in vivo.

Pancreas-on-a-chip mimics in situ pancreatic cell function and interface compared to conventional human cell culture model. The chip allows mimicking of fluid flow in vivo by setting a perfusion system in a cell culture incubator or on a microscope, relevant mechanical cues in cellular signaling, and allows tissue-issue interface (i.e., duct-islet) to study cell-cell signaling³⁰. Pancreas-on-a-chip helps answer the fundamental question in CFRD: is loss of CFTR function in PDECs primary to CI-RD development. Based on the data, it is indeed the case. Surprisingly, the absolute amount of insulin was around 50% decreased during inhibition of CFTR channel function. In the human pancreas, the organ system is extremely complex in physiological and pathological perspectives. However, Applicant has found that CFTR channel function plays an important role in maintaining endocrine function and may provide insight into the etiology of CFRD. To investigate the crosstalk between PDECs and pancreatic islets, metabolism studies of these two cell types may be performed. CFRD is a serious complication in CF patients who in general have disordered glucose metabolism with increasing risk with advancing age.

Using this in vitro chip model, CFRD and glucose imbalance can be studied in CF individuals, assay variability in the glucose measures in these individuals, determine correlation of glucose levels with the CFTR mutation type, and test small-molecule interventions (i.e., approved CFTR modulators) that may improve glucose abnormalities in the patient samples. Applicant's data based on the effect of CFTR-specific inhibitor and lack of function mutation in CFTR strongly suggests that CFTR function modulates insulin secretion that underlies the pathology of CFRD. This patient-derived in vitro model system also allows the development of personalized medicine with highly sensitive measurements of epithelial and/or endocrine functions from the pancreatic cells. Because the cells cultured in the chip are all patient derived, Applicant can easily and quickly obtain other clinically relevant measures using this model in safe manner Alcohol abuse has been reported to lead to dysfunction and degradation of CFTR protein on the apical membrane of the epithelium³¹. Using this chip model, Applicant can monitor CFTR function and/or endocrine function in response to alcohol in a dose-dependent manner that is not possible in patients. The microfluidic device can be set up for multiple analyses, including functional assays and microscopic measurements in real time. This in vitro model system will facilitate drug discoveries. However, the polydimethylsiloxane (PDMS) used for the cell culture chambers has a challenging property, which is that the hydrophobic PDMS absorbs hydrophobic small molecules^{32,33}. After oxygen plasma treatment, it changes to highly hydrophilic³⁴. However, it recovers to hydrophobic over time³⁵. The hydrophobic surface interferes with cell adhesion on the substrate³⁶. Alternatively, other materials for fabricating the micro-fluidic device have been adopted, such as poly methyl methacrylate³⁷, acrylonitrile butadiene styrene copolymer³³, cyclic olefin copolymer³⁸, and styrene ethylene butylene styrene³⁹. However, those materials also have limitations when mimicking human organ systems due to their rigidity and brittleness, leading to a difficult fabrication process. PDMS-based microfluidic devices can be maintained as a hydrophilic surface for weeks after treatment with oxygen plasma³⁴. It has also been shown that sol-gel-modified PDMS⁴⁰ and bovine serum albumin-coated-PDMS⁴¹ can minimize absorbance of hydrophobic drugs by PDMS. Alternatively, collagen coating of the chamber can be utilized to increase cell adhesion, as is used in the model system.

In summary, Applicant has isolated and cultured patient-derived pancreatic cells, PDECs, and pancreatic islets from the same patient. This efficient and highly reproducible method allows the study of pancreatic disorders. Moreover, the in vitro model system, pancreas-on-a-chip, allows for the investigation of the crosstalk between PDECs and islet cells in the development of disease pathologically and physiologically. This pancreas-on-a-chip model system, with its highly sensitive profile, can allow for early diagnosis and individual diagnosis that may help prevent or reduce the progression of disorders such as CFRD, and additionally, can afford the opportunity for drug discovery and personalized medicine in such disorders.

Methods

Human Studies.

Human tissue, pancreatic remnant cell pellets were collected according to standard research protocols approved by the Institutional Review Board and Department of Pathology at Cincinnati Children's Hospital (IRB: 2014-6279; renewed 27 Nov. 2017).

Cell Culture Media.

For PDECs, advanced Dulbecco's modified Eagle's medium/nutrient mixture F12 (DMEM/F12) (Invitrogen; Ser. No. 12/634,010) with 10 mM HEPES (Invitrogen; #15630-080), GlutaMAX (lx; Invitrogen; #35050-061), and penicillin streptomycin (PS) (lx; Invitrogen; #15140-122) was used as base organoid media(I). Organoid media (II) contains N2 (lx; Invitrogen; #17502-048), B27 (lx; Invitrogen; #17504-044), and 1 mMN-acetylcysteine (Sigma; #A7250-100G) in organoid media (I). Organoid media (III) contains growth factors, supplements 100 ng/mL epidermal growth factor (R&D System; #236-EG-200), 50 ng/mL R-spondin (R&D System; #4645-RS-025/CF), and 100 ng/mL Noggin (R&D System; #6057-NG-025) in organoid media (II). ROCK inhibitor, 10 μM Y-27632 (BDBiosciences; #562822), was added for the first 4 days followed by isolation of pancreatic ductal epithelial cells. Islet cells were cultured in RPMI-1640 (Invitrogen; No. 61/870,036) containing PS(lx), 10% fetal bovine serum (FBS) (Atlanta Biologicals; #S11150 premium), and 10 μM Y-27632 for the first day of isolation. The RPMI-1640 media were switched to low glucose-containing DMEM (Invitrogen; #11885-084; 100 mg/dL glucose) with PS (lx) and 10% FBS from the second day of isolation. High-glucose-containing DMEM (Invitrogen; #11960-044; 450 mg/dL glucose) was used to stimulate pancreatic islets.

Isolation of pancreatic ductal organoids and islet.

Pediatric patients with severe acute recurrent or chronic pancreatitis undergo TPIAT. During the TPIAT, the excised pancreas was surgically dissected and digested to isolate pancreatic islets for infusion into the liver through the portal vein. Applicant obtained discarded pancreatic remnant cell pellets following isolation of pancreatic islets. The pancreatic remnant cell pellet still contains pancreatic islets, ductal epithelial cells, and acinar cells (FIG. 1C). From the remnant cell pellet, Applicant isolated pancreatic ductal tissues by microscopic dissection under a stereo microscope (Leica; #M165FC) (FIG. 1e). The pancreatic remnant cell pellet was prepared on a 60-mm dish containing phosphate-buffered saline (PBS) by dissecting a white cluster (FIG. 1B, FIG. 1D) using two forceps until the appearance of pancreatic duct-like structure remained. Then, holding the cluster using one forcep the pancreatic duct is gently pulled out using the other forceps. The pancreatic duct is extracted smoothly, because surrounding connecting tissues were uniformly oriented along the length of the duct. Microdissection scissors were used to remove other cell types attached to the ductal tissues, if necessary. Isolated ductal tissues were treated with 2 mM EDTA (Invitrogen; #15575) in 10 mL PBS on a shaker (Nutator; #421105) at 4° C. for 40 mM The tissues were filtered through a 70 μm strainer (Falcon; #352350) to remove the EDTA solution, and all tissues were transferred to fresh 15 mL tube containing 10 mL PBS. The tube was shaken mechanically to help separate ductal epithelial cells apart from the tissues. The supernatant was then filtered through a fresh 70 μm strainer and 10% FBS was added to stop the proteolytic activity. Supernatant was discarded after centrifugation (Beckman Coulter; #Allegra X-14R) at 233×g for 3 mM Organoid media (II) were added and the ductal epithelial cells were re-suspended by pipetting gently. Matrigel (Corning; #356231) was added at a ratio of 2:3 vol % growth media to Matrigel and mixed well avoiding bubbles. Matrigel (50 μL) was plated with ductal cells on a substrate and incubated at 37° C. for 15 min for solidification of the Matrigel. The Matrigel was covered with 500 μL organoid media (III) containing growth factors. For the first 4 days, 10 μM Y-27632 of ROCK inhibitor was added to help cellular recovery from cellular aggregates^{42,43}. Organoid media (III) were refreshed every other day. Pancreatic remnant cell pellets contain leftover islet cells after isolation of islets for infusion. Islet cells can be easily identified by dithizone staining (100 μg/mL), because dithizone binds to the zinc ion presented in insulin secreted by (kens in the pancreatic islets²⁵. Dithizone solution is green, but it turns to red when it binds to the zinc ion (FIG. 1d, 1g). Islet cells were transferred gently to a fresh plate using a 200 μL pipette and cultured in RPMI-1640 media containing 10 μM Y-27632 for the first day. Next day, the media were switched to low glucose-containing DMEM (100 mg/dL) and refreshed every other day.

Obtaining monolayer of pancreatic ductal epithelial cells.

Pancreatic ductal organoids were grown over time in Matrigel (FIG. 1H). The mechanism is unknown, but Applicant continuously observed that when the ductal organoids contact the surface, they start to form duct-like structures and then a complete monolayer (FIG. 7). To assist forming a monolayer of PDECs, the Matrigel was broken down by pipetting with 1 mL organoid media (I) when the average diameter of the organoids reached 500 μm. Organoids were then transferred to 1.5 mL tube by using a 200 μL pipette. After centrifugation at 8600×g for 3 min (Eppendorf microcentrifuge; #5418), the supernatant was discarded and the organoid media (III) containing 10 μM Y-27632 were added to the pellet. Organoids were then plated on a fresh dish or trans-well membrane. The Matrigel may be separated substantially completely from the organoids, for improved adherence and survival.

Freezing and reviving pancreatic ductal epithelial cells.

To cryopreserve PDECs, PDEC monolayers were trypsinized with 0.5% trypsin EDTA (1×; Invitrogen; #15400-054) at 37° C. for 10 min to detach cells after washing cells with PBS and transferred to 15 mL tube containing 5 mL organoid media (I) with 10% FBS and 10 μM Y-27632. Cell pellets were obtained after centrifuging at 233×g for 5 min. The supernatant was discarded and cells were re-suspended with freezing media (Invitrogen; Ser. No. 12/648,010) containing 10 μM Y-27632. Epithelial cells were transferred to a cryopreservation tube and placed on dry ice immediately and stored at −80° C. For long-term storage, the cells were stored in liquid nitrogen. To revive PDECs, the cells were thawed quickly at 37° C. and all supernatants were transferred to 15 mL tube containing 5 mL organoid media (I) with 10 μM Y-27632. After centrifugation at 233×g for 5 min, the supernatant was discarded. Appropriate organoid media (II) with Matrigel were added and 50 μL Matrigel was plated with cells on plates to form organoid structure as before. The Matrigel was covered with organoid media (III) containing 10 μM Y-27632 after incubation at 37° C. for 15 min.

Fabrication of Pancreas-On-a-Chip.

Applicant's customized microfluidic device was designed to mimic ductal structure having branches with narrowing diameters (FIG. 10). The design was drawn using the AutoCAD software. The chip was fabricated through the standard photolithography and soft lithography techniques (FIG. 11). Initially, the silicon wafer was washed with acetone, isopropanol (IPA) and water. It was placed on a hot plate at 60° C. for 10 min to dry thoroughly after air drying.

After cooling down to room temperature, a negative photoresist SU-8 (Microchem; #Y131269) is applied to the wafer using a spin coater (Specialty Coating Systems; #6800) by the following process: (1) Place the wafer on the vacuum chuck of the spin coater and drop appropriate SU-8 on the wafer. (2) Ramp up to 500 rpm for 10 s and hold for 10 s. (3) Increase the speed to 1000 rpm for 10 s and hold it for 15 s for 140 μm thickness of cell culturing chamber in the chip. (4) Speed down to 0 rpm for 10 seconds. The wafer is placed on the hot plate and baked at 65° C. for 10 min and at 95° C. for 30 min serially. The wafer is exposed to ultraviolet (UV) light (wavelength: 365 nm; exposure energy:240mJ/cm2) through a patterned photomask for 20 s after cooling down to room temperature. The wafer is baked on the hot plate at 65° C. for 1 min and at 95° C. for 20 min and is cooled down to room temperature. The wafer is immersed into SU-8 developer (Fisher Scientific; #NC9901158) for development process of unexposed area to UV light. After completion of development, the wafer is washed with IPA and dried with filtered air. The patterned silicon wafer is then baked on the hot plate at 150° C. for 30 min After cooling down to room temperature, the patterned wafer can be used as a mold. These standard photo-lithography procedures can be carried out in in a 100-class clean room. For this microfluidic device, Applicant used flexible, transparent, and low-cost materials, PDMS (Ells Worth Adhesive; #4019862). Viscous PDMS is mixed with a curing kit at the ratio of 10:1 (wt %) and degassed in a desiccator to remove bubbles. In the meantime, the patterned silicon wafer is treated with trichloro silane (Sigma-Aldrich; #448931) for 30 min in another desiccator to assist peeling off the patterned PDMS layer from the wafer. The uncured PDMS is cast onto the wafer and cured at 60° C. for at least 4 h. The solidified patterned PDMS layer is peeled off from the wafer and holes are created at both ends of the cell culture area for seeding and feeding cells. The patterned PDMS layer and a cover glass are treated with oxygen plasma for 30 s using Tergeo Plasma Cleaner (PIE Scientific) and immediately assembled together. It is placed on the hot plate at 120° C. for 30 min to seal completely the single-channel chip. The activated surface of the patterned PDMS layer and cover glass by plasma treatment becomes highly hydrophilic with polar characteristics³⁴. This enhances the bonding process of the two surfaces. Pancreas-on-a-chip is comprised of top and bottom layers for cell culture chambers and a thin layer of porous membrane to separate the two chambers as double-channel chip. Patterned PDMS layers of top and bottom chambers are prepared as described previously for single-channel chip. Holes were created through the PDMS layer of the top chamber for seeding and feeding cells before assembly with the porous membrane. For the thin layer of porous membrane, a mold was fabricated of uniformly arranged cylinders, with 10 μm diameters, 25-μm gaps, and 40-μm thickness, on a silicon wafer through the photolithography. The wafer is coated with trichloro silane in the desiccator for 30 min. In the meantime, RTV615 (Momentive; #9480), which shows large linear behavior of stain and promotes fabrication of a thin layer uniformly comparing to PDMS^{44,45}, is mixed with a curing kit at the ratio of 5:1 (wt %) and degassed in the desiccator for 30 min. The patterned wafer was placed on the spin coater and spun after covering the pattern with degassed RTV615 as the standard for 10 μm thickness of porous membrane; thus, (1) ramp up to 500 rpm for 10 s and hold for 10 s; (2) increase the speed to 3000 rpm for 10 s and hold it for 5 min; (3) speed down to 0 rpm for 10 s. Leave the wafer at room temperature for 10 min for uniform surface and incubate at 60° C. for 10 min for partial solidification of the surface. After incubation, the top PDMS layer was placed, patterned face down, directly onto the cylinders and slightly pressed onto the PDMS layer for contacting the surface of top layer to the partially cured RTV615. The top chamber is incubated with the porous membrane on the wafer overnight and cooled down to room temperature. The top chamber with the porous membrane is peeled from the wafer and holes created through the porous membrane to connect to the bottom chamber only. The top chamber and bottom chamber are aligned after oxygen plasma treatment and placed on the hotplate at 120° C. for 30 min to seal the double-channel chip. Before seeding cells, the cell culture chambers were sterilized with 70% EtOH for 10 min and washed with autoclaved water using a needle (BD Biosciences; #305175; 20 G) and syringe (BD Biosciences; #309657; 3 mL). The chambers were coated with 50 μg/mL collagen (Sigma-Aldrich; #C3867-1VL) for 1 h at 37° C. and washed with PBS to increase cell adhesion. The microfluidic device was connected to a peristaltic pump (Cole-Parmer; #ISMATEC Reglo ICC) with tubing (Cole-Parmer; #97619-09) and supplied organoid growth media (III) at the flow rate of 1 μL/min to feed cells continuously. To feed cells manually, a syringe and needle was used.

Culture Cells in the Microfluidic Device.

Monolayers of PDECs were treated with 0.5% Trypsin EDTA (lx) at 37° C. for 10 min and floating cells were transferred to a 15 mL tube containing 5 mL organoid media (I) with 10% FBS and 10μMY-27632. The supernatant was discarded after spinning down at 233×g for 5 min (4° C.) and cells were re-suspended with 120 μL organoid media (III) containing 10 μM Y-27632. Cells were transferred (2×10⁵ cells/mL; 10,000 cells/chip) in the cell culture chamber coated with collagen (50 μg/mL) using a syringe and needle through a tubing (5 cm length) inserted through the PDMS layer. After overnight incubation at 37° C., 5% CO2 media were refreshed. Pancreatic islets in 24-well plate were washed with PBS and incubated with 200 μL 0.5% Trypsin EDTA (lx) at 37° C. for 3 min for trypsinization. Pancreatic islets were transferred to a 1.5 mL tube filled with culture media. The supernatant was discarded after centrifugation at 8600×g (microcentrifuge) for 3 min and cells were re-suspended with 120 μL media. Pancreatic islets were transferred (300 islets/mL; 15 islets/chip) into the cell culture chamber using a syringe and needle. Media were refreshed after the pancreatic islets attached onto the surface of the chip.

Immunofluorescence Microscopy.

Pancreatic ductal organoids in Matrigel were fixed with 3.7% formaldehyde for 15 min at room temperature and the Matrigel was broken down by pipetting with 1 mL EtOH. The organoids were embedded into HistoGel (Invitrogen; #HG-4000-012) and were first examined by gold-standard morphological section and H&E stain. Paraffin-sectioned organoids were deparaffinized for immunofluorescence microscopy. For a monolayer of PDECs on a trans-well membrane or a pancreas-on-a-chip, cells were fixed with 3.7% for maldehyde for 15 min at room temperature. Cells were then permeabilized using lx permeabilization solution (eBioscience; #00-8333-56) for 8 min at room temperature and washed three times with PBS for 5 min each. Cells were then blocked using 1% goat serum (Sigma-Aldrich; #A8806-5G) for 1 h at room temperature and incubated with primary antibodies (diluted in antibody diluent (Invitrogen; #TA-125-ADQ) 1:100), anti-CFTR R1104 (Eric Sorscher lab, CF Center, University of Alabama, Birmingham, Ala., USA [presently, Emory University, Atlanta, Ga., USA]), anti-ZO-1 (BD Biosciences; #610967), anti-ENaC (Invitrogen; #PA1-920A), anti-KRT 19 (Invitrogen; #MA5-12663), anti-E cadherin (Cell Signaling Technology; #3195), anti-insulin (Cell Signaling; #C27C9), and anti-glucagon (Sigma; #G2654) overnight at 4° C. Cells were washed three times with PBS for 5 min each and incubated with secondary antibodies (Invitrogen; Alexa Fluor 488 or 568; 1:500) for 1 h at room temperature. Alexa Fluor 488 Phalloidin (Invitrogen; #A12379; 1:50) was employed to the secondary antibody for F-actin staining fol-lowing washing three times with PBS for 5 min each. Cells were incubated with DAPI solution (Invitrogen; #D1306; 1:500) for 20 min for nucleus staining and washed with PBS. For the trans-well membrane, cut edge of the membrane and transferred the membrane with cells onto a glass slide oriented cell-side up. Cells were then mounted in Vecta-shield mounting medium (Vector Labs; #H-1000). A cover slip was placed onto the cells and fixed with nail polish. For the pancreas-on-a-chip, cell culture chambers were separated manually by hands followed by nuclear staining with 4′,6-diamidino-2-phenylindole (DAPI) solution for 20 min. The porous membrane with cells remained on the upper layer. One drop of mounting solution was applied onto the cells and a coverslip was placed for imaging. Fluorescence images were obtained using a confocal microscope (Olym-pus FV1200). Combined images were created using an Image J software provided by NIH.

Extract RNA from Pancreatic Ductal Organoids.

Organoid growth media were discarded and the Matrigel was broken down by pipetting with 1 mL PBS. Pancreatic ductal organoids were picked and transferred to 1.5 mL RNA-free tube manually using a 200 μL pipette. The supernatant and Matrigel were discarded after microcentrifuge at 16,800×g for 5 min and RNA was extracted using an Ambion miRNA Isolation Kit (Invitrogen; #AM1561) using the protocol provided by Ambion.

Monitoring CFTR Function.

CFTR function of pancreatic ductal organoids was monitored using the fluid secretion assay²⁶ in response to an intracellular cAMP-activating agonist (FSK; 10 μM) for 2 h at 37° C. Fluid secretion was calculated by measuring the volume ratio of luminal area over the entire organoid pre-treatment and post treatment with FSK. Fluid secretions were monitored at day 4 after isolation of organoids with at least 20 organoids. The area of lumen and outer sphere was measured using the Image J software. Pancreatic ductal organoids were transferred, when their diameter reached 500 μm, onto trans-well membranes (Corning; #3470), 10 organoids each as previously described. The ductal epithelial cells transformed into a polarized monolayer from spheroids on the trans-well membrane within 2 weeks. Transepithelial electrical resistance was measured using epithelial volt-ohm meter (World Precision Instruments, #EVOM and #STX2) and the trans-well membrane was mounted in an Ussing chamber when the resistance was over 1000 Ω/cm2. Cells were bathed in Ringer's solution (mM) for apical side (pH 7.2): 0.12 NaCl, 25 NaHCO₃,3.3 KH₂PO₄, 0.83 KH₂PO₄, 1.2 CaCl₂, 1.2 MgCl₂, 141 Na-gluconate, and 10 mannitol, and for basolateral side (pH 7.2): 120 NaCl, 25 NaHCO₃, 3.3 KH₂PO₄, 0.83 KH₂PO₄, 1.2 CaCl₂, 1.2 MgCl₂, and 10D-glucose maintained the temperature of the bath using circulate system as 37° C.^{26,46}. CFTR function was monitored in real time in response to current changing by FSK. When the current showed a stable baseline, 10 μM FSK was added to the apical side for CFTR channel opening. For CFTR channel closing, CFTR channel inhibitor, CFTRᵢₙₕ₋₁₇₂ (20 μM), was applied to the apical side. CFTR function of PDECs was monitored using iodide efflux assay⁴⁷. Cell culture media were washed out with 136 mM NaNO₃ and incubated with 136 mMNaI for 1 h at 37° C. After 1-h incubation, cells were washed with 300 μL of NaNO₃ (136 mM) and the supernatant was collected with 136 mM NaNO₃ using a syringe and needle. A 1.5 mL tube was placed on a digital weighing scale and the supernatant was dropped into the tube with recording the weight for approximately 20 μL of each sample. The first 10 samples were collected with 136 mM NaNO₃ and the other 10 samples with 136 mM NaNO₃ containing 10 μMFSK. Iodide concentration was calculated using an electrolyte detector (Thermo Orion; #420) with electrode probe filled with specific iodide-sensitive electrolyte (Invitrogen; #900063). The electrode was immersed in 5 mL of 100 mM NaNO₃ (stirred) to detect iodide. Voltage change was measured by adding each sample serially. A standard curve was obtained using 10 μM, 100 μM, and 1 mM NaI.

Monitoring Insulin Secretion.

Cell culture media were discarded just before collection for the measurement. Media (60 μL) were collected and incubated with refreshed media for 1 h at 37° C., 5% CO2. After 1-h incubation, an additional 60 μL media were collected. Collected media were placed on ice until ready to assay. For stimulation of pancreatic islets, 450 mg/dL glucose-containing media (instead of 100 mg/dL) were used. Insulin secretion was monitored by measuring concentration of insulin in the culture media using ELISA (Invitrogen; #KAQ1251) following a protocol provided by the company. To monitor insulin secretion from pancreas-on-a-chip, Applicant co-cultured PDECs in the top chamber and pancreatic islets in the bottom chamber. Base media for PDECs, advanced DMEM/F12, contains insulin, which can affect the concentration of insulin secreted by pancreatic islets in the bottom chamber. It was switched to DMEM (same as pancreatic islets media). Two chips were prepared, Chip A and Chip B, to employ agonist (10 μM FSK) or inhibitor (20 μM CFTRᵢₙₕ₋₁₇₂) of CFTR channel on the PDECs. The chips were incubated at 37° C. for 1 h and 60 μL media were collected from the bottom chamber. FSK (Chip A) and CFTRᵢₙₕ₋₁₇₂ (Chip B) were employed on the top chambers and the chips were incubated at 37° C. for 1 h. Sixty microliters of media was collected from the pancreatic islets on the bottom chamber. For Chip A, the media in the bottom chamber were switched to high-glucose-containing media (450 mg/dL) and the chip was incubated at 37° C. for 1 h. For Chip B, a combination of FSK and CFTRᵢₙₕ₋₁₇₂ were added to the top chamber and the chip was incubated at 37° C. for 1 h. The chip was incubated with high-glucose-containing media at 37° C. for 1 h. Sixty microliters of media were collected from the pancreatic islets in the bottom chamber.

Statistical Analysis.

Data were derived from at least three independent replicates. The level of marginal significance, p-value, was calculated using two-tailed Student's t test for pairwise comparison and one-way analysis of variance with Bonferroni adjustment for multiple variations. A p value <0.05 was considered significant. Reporting summary

Exemplary Pancreatic Microfluidic Device

FIGS. 16-24 show one example of the microfluidic device (10) for use as the pancreas-on-a-chip as discussed above in greater detail. With respect to FIG. 16-18, the microfluidic device (10) includes an upper plate (12), a lower plate (14), and a porous, permeable membrane (16) sandwiched therebetween. The upper and lower plates (12, 14) respectively include an upper inner surface (16) and a lower inner surface (18). The upper inner surface (16) at least partially defines a top chamber (22) that receives a plurality of PDECs (24), whereas the lower inner surface (20) at least partially defines a bottom chamber (26) that receives a plurality of pancreatic islets (28). The top chamber (22) is positioned transversely opposite and offset from the bottom chamber (26) with the permeable membrane (16) positioned therebetween thereby fluidly connecting the top and bottom chambers (22, 26). In turn, the plurality of PDECs (24) in the top chamber (22) is configured to communicate with the plurality of pancreatic islet in the bottom chamber (26) for mimicking in situ pancreatic cell function.

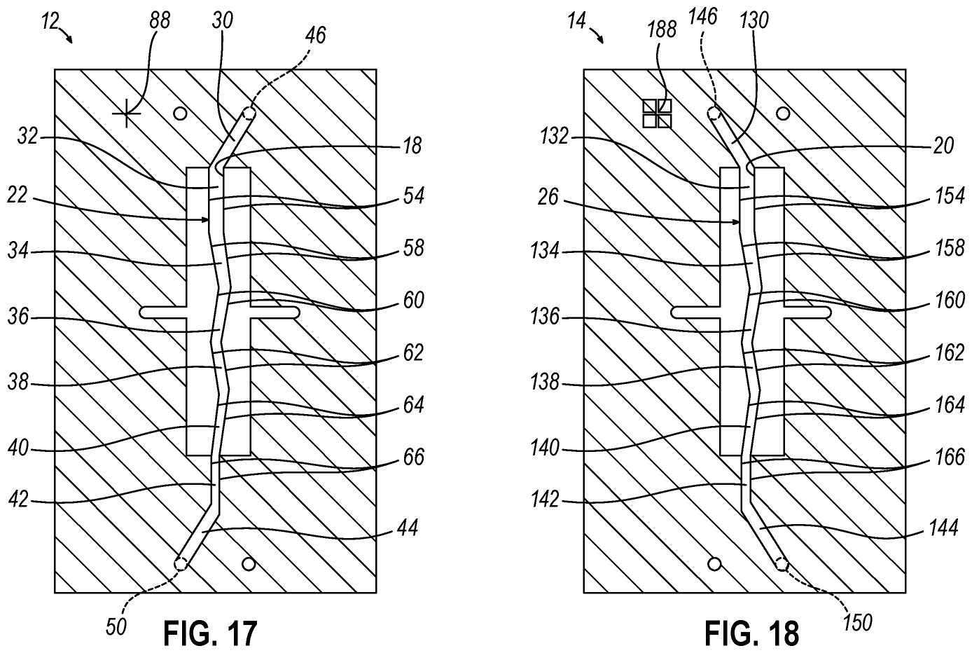

As shown in FIGS. 16 and 17, the upper plate (12) more particularly has the upper inner surface (18) defining the top chamber (22). To this end, the top chamber (22) of the present example includes a first end top channel (30), a first top branch channel (32), a second top branch channel (34), a third top branch channel (36), a fourth top branch channel (38), a fifth top branch channel (40), a sixth top branch channel (42), and a second end top channel (44) extending through the upper plate (12) in a common plane of the upper plate (12). The first end top channel (30) is longitudinally opposite of the second end top channel (44) and fluidly connected by the first, second, third, fourth, fifth, and sixth top branch channel (32, 34, 36, 38, 40, 42) successively fluidly connected therebetween. The first end top channel (30) intersects a first top hole (46), which transversely extends through the upper plate (12) and further fluidly connects to a first top tubular conduit (48) for seeding and feeding cells as discussed above. Similarly, the second end top channel (44) intersects a second top hole (50), which transversely extends through the upper plate (12) and further fluidly connects to a second top tubular conduit (52) for further seeding and feeding cells as discussed above.

In order to more effectively mimic pancreatic duct-like structures as discussed above, the top chamber (22) successively narrows from the first top end channel (30) toward the second top end channel (44) at each of the first, second, third, fourth, and fifth top branch channels (32, 34, 36, 38, 40). More particularly, the first top branch channel (32) includes a first pair of top edges (54) extending in the common plane of the upper plate (12) and defining a first top width therebetween. Similarly, the second, third, fourth, and fifth, and sixth top branch channels (34, 36, 38, 40, 42) respectively include second, third, fourth, fifth, and sixth pairs of top edges (58, 60, 62, 64, 66) and respectively define second, third fourth, fifth, and sixth top widths. The first, second, third, fourth and fifth top widths successively narrow such that the the fifth top width is smaller than the fourth top width, the fourth top width is smaller than the third top width, the third top width is smaller than the second top width, and the second top width is smaller than the first top width. As used herein, the term “edges” generally refers to opposing sides of a channel of top chamber (22) that define a width therebetween, such as any one or more of top branch channels (32, 34, 36, 38, 40, 42), and is not intended to unnecessarily limit the invention described herein.

In addition, the first, second, third, fourth, and fifth top branch channels (32, 34, 36, 38, 40, 42) of the present example also respectively include first, second, third, fourth, fifth, and sixth depths that are respectively equal to the first, second, third, fourth, fifth, and sixth top widths. In instances where the depths are widths of respective top branch channels are equal, the first, second, third, fourth, fifth, and sixth top widths may also be referred to as first, second, third, fourth, fifth, and sixth top diameters. The first, second, third, fourth, fifth, and sixth depths respectively extend from the first, second, third, fourth, fifth, and sixth pairs of top edges (54, 58, 60, 62, 64, 66) to a top chamber floor, portions of which may be generally flat between the first, second, third, fourth, fifth, and sixth pairs of top edges (54, 58, 60, 62, 64, 66) and/or curved between the first, second, third, fourth, fifth, and sixth pairs of top edges (54, 58, 60, 62, 64, 66).

Also in order to more effectively mimic pancreatic duct-like structures as discussed above, each of the first, second, third, fourth, and fifth top branch channels (32, 34, 36, 38, 40, 42) intersects adjacent top branch channels (32, 34, 36, 38, 40, 42) as applicable at predetermined angles. In this respect, the first and second top branch channels (32, 34) intersect at a first top predetermined angle, the second and third top branch channels (34, 36) intersect at a second top predetermined angle, the third and fourth top branch channels (36, 38) intersect at a third top predetermined angle, the fourth and fifth top branch channels (38, 40) intersect at a fourth top predetermined angle, and the fifth and sixth top branch channels (40, 42) intersect at a fifth top predetermined angle. As used herein, “predetermined angle” refers to an angle that is neither 0 degrees nor 180 degrees such that adjacent first, second, third, fourth, and fifth top branch channels (32, 34, 36, 38, 40, 42) are non-parallel relative to each other, although non-adjacent top branch channels (32, 34, 36, 38, 40, 42) may be parallel in some examples. In the present example, the first, second, third, fourth, and fifth top predetermined angles are oriented such that the first, second, third, fourth, and fifth top branch channels (32, 34, 36, 38, 40, 42) laterally zigzag back and forth while also longitudinally projecting from the first end top channel (30) to the second end top channel (44) such that the first and sixth top branch channels (32, 42) are parallel to each other.

Similar to the upper plate (12), FIGS. 16 and 18 also show that the lower plate (14) more particularly has the lower inner surface (20) defining the bottom chamber (26). To this end, the bottom chamber (26) of the present example includes a first end bottom channel (130), a first bottom branch channel (132), a second bottom branch channel (134), a third bottom branch channel (136), a fourth bottom branch channel (138), a fifth bottom branch channel (140), a sixth bottom branch channel (142), and a second end bottom channel (144) extending through the lower plate (14) in a common plane of the lower plate (14). The first end bottom channel (130) is longitudinally opposite of the second end bottom channel (144) and fluidly connected by the first, second, third, fourth, fifth, and sixth bottom branch channel (132, 134, 136, 138, 140, 142) successively fluidly connected therebetween. The first end bottom channel (130) intersects a first bottom hole (146), which transversely extends through the lower plate (14) and further fluidly connects to a first bottom tubular conduit (148) for seeding and feeding cells as discussed above. Similarly, the second end bottom channel (144) intersects a second bottom hole (150), which transversely extends through the lower plate (14) and further fluidly connects to a second bottom tubular conduit (152) for further seeding and feeding cells as discussed above.

In order to more effectively mimic pancreatic duct-like structures as discussed above, the bottom chamber (26) successively narrows from the first bottom end channel (130) toward the second bottom end channel (144) at each of the first, second, third, fourth, and fifth bottom branch channels (132, 134, 136, 138, 140). More particularly, the first bottom branch channel (132) includes a first pair of bottom edges (154) extending in the common plane of the lower plate (14) and defining a first bottom width therebetween. Similarly, the second, third, fourth, fifth, and sixth bottom branch channels (134, 136, 138, 140, 142) respectively include second, third, fourth, fifth, and sixth pairs of bottom edges (158, 160, 162, 164, 166) and respectively define second, third fourth, fifth, and sixth bottom widths. The first, second, third, fourth, and fifth bottom widths successively narrow such that the the fifth bottom width is smaller than the fourth bottom width, the fourth bottom width is smaller than the third bottom width, the third bottom width is smaller than the second bottom width, and the second bottom width is smaller than the first bottom width. Again, as used herein, the term “edges” generally refers to opposing sides of a channel of bottom chamber (26) that define a width therebetween, such as any one or more of bottom branch channels (132, 134, 136, 138, 140, 142), and is not intended to unnecessarily limit the invention described herein.

In addition, the first, second, third, fourth, and fifth bottom branch channels (132, 134, 136, 138, 140, 142) of the present example also respectively include first, second, third, fourth, fifth, and sixth depths that are respectively equal to the first, second, third, fourth, fifth, and sixth bottom widths. In instances where the depths are widths of respective bottom branch channels are equal, the first, second, third, fourth, fifth, and sixth bottom widths may also be referred to as first, second, third, fourth, fifth, and sixth bottom diameters. The first, second, third, fourth, fifth, and sixth depths respectively extend from the first, second, third, fourth, fifth, and sixth pairs of bottom edges (154, 158, 160, 162, 164, 166) to a bottom chamber floor, portions of which may be generally flat between the first, second, third, fourth, fifth, and sixth pairs of bottom edges (154, 158, 160, 162, 164, 166) and/or curved between the first, second, third, fourth, fifth, and sixth pairs of bottom edges (154, 158, 160, 162, 164, 166).

Also in order to more effectively mimic pancreatic duct-like structures as discussed above, each of the first, second, third, fourth, and fifth bottom branch channels (132, 134, 136, 138, 140, 142) intersects adjacent bottom branch channels (132, 134, 136, 138, 140, 142) as applicable at predetermined angles. In this respect, the first and second bottom branch channels (132, 134) intersect at a first bottom predetermined angle, the second and third bottom branch channels (134, 136) intersect at a second bottom predetermined angle, the third and fourth bottom branch channels (136, 138) intersect at a third bottom predetermined angle, the fourth and fifth bottom branch channels (138, 140) intersect at a fourth bottom predetermined angle, and the fifth and sixth bottom branch channels (140, 142) intersect at a fifth bottom predetermined angle. Again, as used herein, “predetermined angle” refers to an angle that is neither 0 degrees nor 180 degrees such that adjacent first, second, third, fourth, and fifth bottom branch channels (132, 134, 136, 138, 140, 142) are non-parallel relative to each other, although non-adjacent bottom branch channels (132, 134, 136, 138, 140, 142) may be parallel in some examples. In the present example, the first, second, third, fourth, and fifth bottom predetermined angles are oriented such that the first, second, third, fourth, and fifth bottom branch channels (132, 134, 136, 138, 140, 142) laterally zigzag back and forth while also longitudinally projecting from the first end bottom channel (130) to the second end bottom channel (144) such that the first and sixth bottom branch channels (132, 142) are parallel to each other.

With continued reference to FIGS. 16-18, a majority of top and bottom chambers (22, 26) are transversely offset from each other so as to overlap in the transverse direction. More particularly, the first top and bottom branch channels (32, 132) have like widths and align in the transverse direction so as to be geometrically the same. Similarly, the second top and bottom branch channels (34, 134) have like widths and align in the transverse direction, the third top and bottom branch channels (36, 136) have like widths and align in the transverse direction, the fourth top and bottom branch channels (38, 138) have like widths and align in the transverse direction, the fifth top and bottom branch channels (40, 140) have like widths and align in the transverse direction, and the sixth top and bottom branch channels (42, 142) have like widths and align in the transverse direction. In contrast to the branch channels (32, 34, 36, 38, 40, 42, 132, 134, 136, 138, 140, 142), the first end top channel (30) and first end bottom channel (130) respectively extend from the first top branch channel (32) and the first bottom branch channel (132) in laterally opposing directions so as to provide clearance for accessing via first top and bottom tubular conduits (47, 147). Similarly, second end top channel (44) and second end bottom channel (144) respectively extend from the sixth top branch channel (42) and the sixth bottom branch channel (142) in laterally opposing directions so as to provide clearance for accessing via second top and bottom tubular conduits (52, 1152). Each of the upper and lower plates (12, 14) further respectively includes upper and lower alignment indicia (88, 188) configured to transversely overlap in a predetermined orientation in order to provide visual feedback that the top and bottom chambers (22, 26) are aligned as shown and described herein.

Each of the portions of the upper and lower plates (12, 14) respectively defining the top and bottom chambers (22, 26) is formed of polydimethylsiloxane (PDMS) although alternative materials may be used to at least some extent as discussed herein. Upon oxygen plasma treatment, the PDMS becomes hydrophobic such that the upper and lower inner surfaces (18, 20) are hydrophobic surfaces. Absorption of hydrophobic drugs may be further reduced by modifying these upper and lower inner surfaces (18, 20) with sol-gel, bovine serum albumin, and/or collagen as further discussed above.

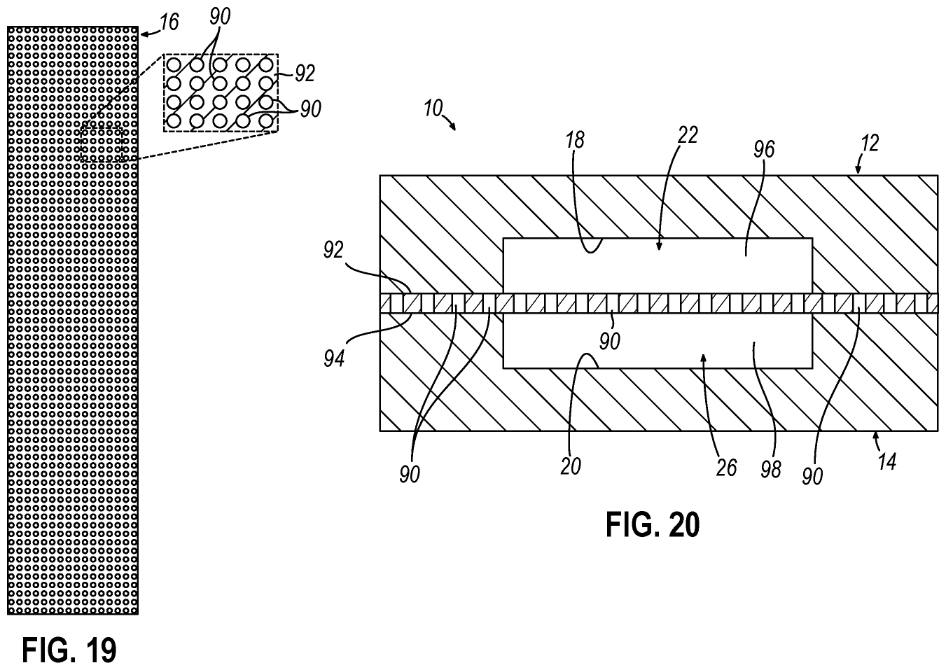

FIG. 19 shows the porous membrane (16) of the microfluidic device (10) (see FIG. 16). The porous membrane (16) of the present example includes a plurality of openings (90) extending therethrough from a top membrane surface (92) to a bottom membrane surface (94). With respect to FIGS. 19-20, each of the plurality of openings (90) fluidly connects the top chamber (22) to the bottom chamber (26) and is configured to allow communication between the plurality of PDECs (24) in the top chamber (22) and the plurality of pancreatic islets (28) in the bottom chamber (26). Each opening (90) in the present example has a diameter from about 5 μm to about 25 μm, including, more particularly, about 10 μm. Furthermore, in the present example, a center of each opening (90) is spaced approximately 25 μm from a center of an adjacent opening (90). It will be appreciated, however, that the invention is not intended to be unnecessarily limited to the particular size and spacing of openings (90) shown and described herein.

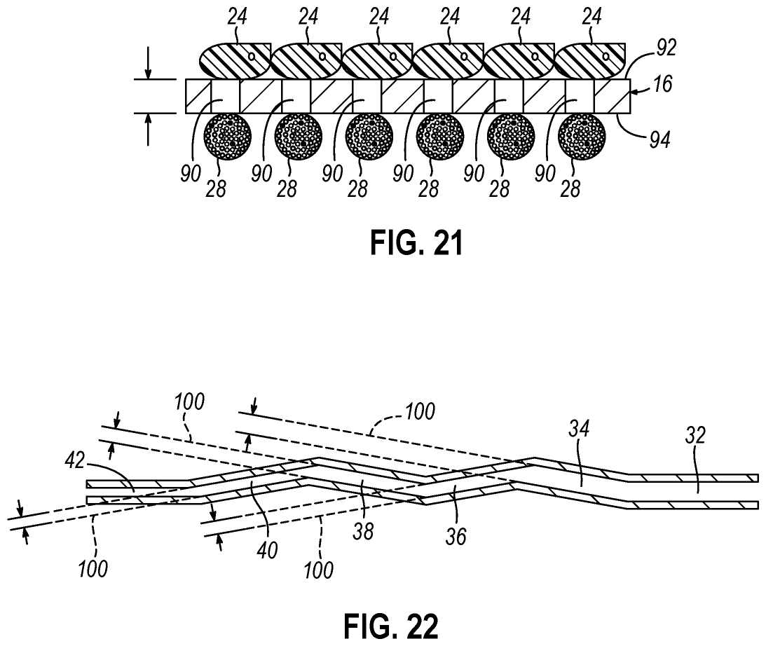

With respect to FIGS. 20 and 21, an upper cell culture media (96) is also contained in the top chamber (22) and may be configured for the cellular material therein, such as the plurality of PDECs (24). Similarly, a lower cell culture media (98) is also contained in the bottom chamber (26) and may be configured for the cellular material therein, such as the plurality of pancreatic islets (28). In one example, the upper cell culture media (96) is the same media materials as the lower cell culture media (98), whereas, in another example, the upper cell culture media (96) is a different media material than the lower cell culture media (98). The invention is thus not intended to be unnecessarily limited to any particular media materials.

FIG. 21 shows the porous membrane (16) transversely between the top and bottom chambers (22, 26), which respectively contain the plurality of PDECs (24) and the plurality of pancreatic islets (28). The porous membrane (16) of the present example has a thickness in the transverse direction of less than approximately 10 μm. In any case, the plurality of PDECs (24) and the plurality of pancreatic islets (28) are configured to communicate through the porous membrane (16) for mimicking in situ pancreatic cell function as described above in greater detail. In one example, the plurality of PDECs (24) and the plurality of pancreatic islets (28) are derived from an individual, such as from the same individual. More particularly, the plurality of PDECs (24) and the plurality of pancreatic islets (28) are derived from the same individual undergoing TPIAT and/or having CI RD, chronic pancreatitis, or acute recurrent pancreatitis.

While the present examples of top and bottom chambers (22, 26) have a variety of channels (30, 32, 34, 36, 38, 40, 42, 44, 130, 132, 134, 136, 138, 140, 142, 144), such as 16 distinct channels, that successively narrow and zigzag with predetermined widths and angles, it will be appreciated that alternative numbers of such channels may be similarly used and arranged in alternative examples. The invention is thus not intended to be unnecessarily limited to the particular top and bottom chambers (22, 26) shown and described herein.

To this end, more particular details of the present top and bottom chambers (22, 26) are shown in FIGS. 22-24. With respect to FIG. 22, at least a portion of top chamber (22) is shown with the first, second, third, fourth, and fifth top branch channels (32, 34, 36, 38, 40) successively narrowing from right to left. In addition, the second top branch channel (34) extends at the first predetermined angle left with a longitudinal component and upward with a lateral component so as to mimic pancreatic duct-like structures. The third top branch channel (36) extends at the second predetermined angle further left with a longitudinal component and downward with a lateral component so as to further mimic pancreatic duct-like structures. This pattern continues successively for each of the first, second, third, fourth, fifth, and sixth top branch channels (32). In addition, these predetermined angles are configured such that the first, second, third, fourth, fifth, and sixth top branch channels (32) do not intersect projections (100) of “cut-off” branches projected beyond each of the first, second, third, fourth, fifth, and sixth top branch channels (32, 34, 36, 38, 40, 42). While not shown, such predetermined angles would similarly apply to bottom chamber (26) in the present example.

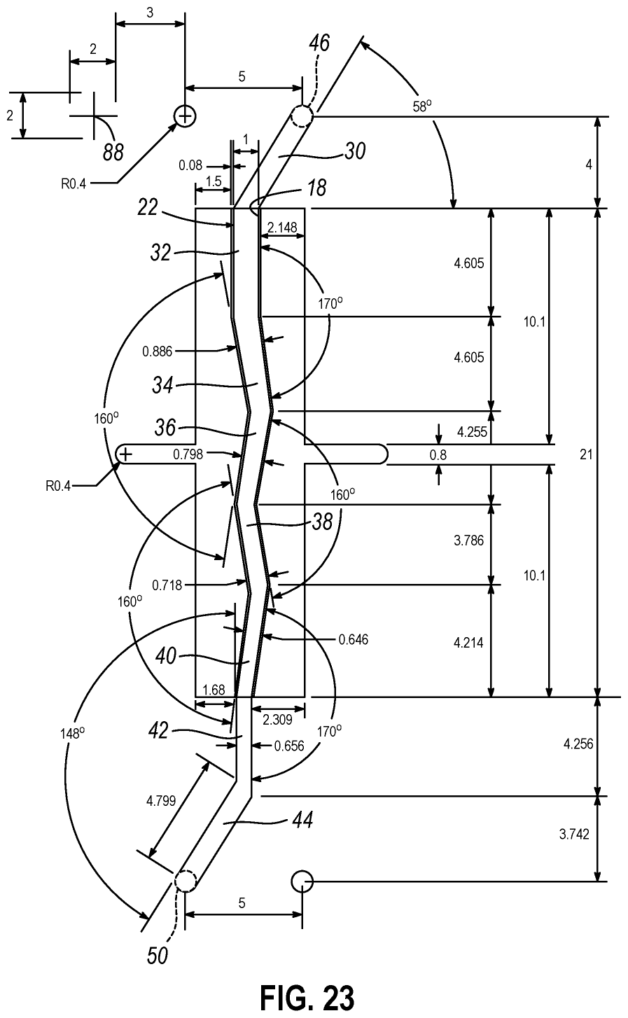

FIG. 23 includes one example of geometries of the top chamber (22) configured to mimic pancreatic duct-like structures. While the invention is not intended to be unnecessarily limited to such geometries, a selection of the particular geometries of the present example are as follows.

| | |
|---|---|
| First Top Width | approximately 1 mm |
| Second Top Width | approximately 0.886 mm |
| Third Top Width | approximately 0.798 mm |
| Fourth Top Width | approximately 0.718 mm |
| Fifth Top Width | approximately 0.646 mm |
| Sixth Top Width | approximately 0.656 mm |
| First Top Predetermined Angle | approximately 170 degrees |
| Second Top Predetermined Angle | approximately 160 degrees |
| Third Top Predetermined Angle | approximately 160 degrees |
| Fourth Top Predetermined Angle | approximately 160 degrees |
| Fifth Top Predetermined Angle | approximately 170 degrees |

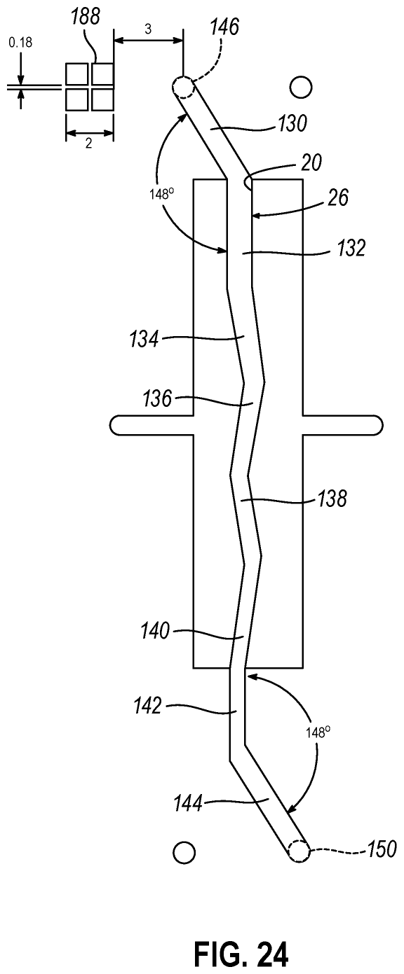

FIG. 24 includes one example of geometries of the bottom chamber (26) configured to mimic pancreatic duct-like structures. Where various dimensions have been omitted and overlap with the top chamber (22) (see FIG. 23), these omitted dimensions are like dimensions to the top chamber (22) (see FIG. 23) and have been omitted merely for greater clarity of differing dimensions. Again, while the invention is not intended to be unnecessarily limited to such geometries, a selection of the particular geometries of the present example are as follows.

| | |
|---|---|
| First Bottom Width | approximately 1 mm |
| Second Bottom Width | approximately 0.886 mm |
| Third Bottom Width | approximately 0.798 mm |
| Fourth Bottom Width | approximately 0.718 mm |
| Fifth Bottom Width | approximately 0.646 mm |
| Sixth Bottom Width | approximately 0.656 mm |
| First Bottom Predetermined Angle | approximately 170 degrees |
| Second Bottom Predetermined Angle | approximately 160 degrees |
| Third Bottom Predetermined Angle | approximately 160 degrees |
| Fourth Bottom Predetermined Angle | approximately 160 degrees |
| Fifth Bottom Predetermined Angle | approximately 170 degrees |

### REFERENCES

1. Marino, C. R., Matovcik, L. M., Gorelick, F. S. & Cohn, J. A. Localization of the cystic fibrosis transmembrane conductance regulator in pancreas. J. Clin. Invest. 88, 712-716 (1991).
2. Zielenski, J. Genotype and phenotype in Cystic fibrosis. Respiration 67, 117-133 (2000).
3. Sheppard, D. N. et al. Mutations in CFTR associated with mild-disease-form Cl-channels with altered pore properties. Nature 362, 160-164 (1993).
4. O'Sullivan, B. P. & Freedman, S. D. Cystic fibrosis. Lancet 373, 1891-1904(2009).
5. Andersen, D. H. Cystic fibrosis of the pancreas and its relation to celiac disease: a clinical and pathologic study. Am. J. Dis. Child. 56, 344-399 (1938).
6. Harutyunyan, M. et al. Personalized medicine in CF: from modulator development to therapy for cystic fibrosis patients with rare CFTR mutations. Am. J. Physiol. Lung Cell. Mol. Physiol. 314, 1529-1543 (2018).
7. Moran, A. et al. Cystic fibrosis-related diabetes: current trends in prevalence, incidence, and mortality. Diabetes Care 32, 1626-1631 (2009).8. Lanng, S. Glucose intolerance in cystic fibrosis patients. Paediatr. Respir. Rev. 2, 253-259 (2001).9. Konrad, K. et al. Cystic fibrosis-related diabetes compared with type 1 and type 2 diabetes in adults. Diabetes Metab. Res. Rev. 29, 568-575 (2013).
10. Marshall, B. C. et al. Epidemiology of Cystic fibrosis-related diabetes. J. Pediatr. 146, 681-687 (2005).
11. Saint-Criq, V. & Gray, M. A. Role of CFTR in epithelial physiology. Cell Mol. Life Sci. 74, 93-115 (2017).12. Strong, T. V., Boehm, K. & Collins, F. S. Localization of Cystic fibrosis transmembrane conductance regulator mRNA in the human gastrointestinal tract by in situ hybridization. J. Clin. Invest 93, 347-354 (1994).
13. Wilschanski, M. & Novak, I. The Cystic fibrosis of exocrine pancreas. Cold Spring Harb. Perspect. Med. 3, https://doi.org/10.1101/cshperspect.a009746(2013).
14. Efrat, S. & Russ, H. A. Makinacells from adult tissues. Trends Endocrinol. Metab 23, 278-285 (2012).
15. Bellin, M. D. et al. Total pancreatectomy and islet autotransplantation in chronic pancreatitis: recommendations from Pancreas Fest. Pancreatology 14, 27-35 (2014).
16. Terry, S. C., Jerman, J. J. & Anyell., J. B. A gas chromatographic air analyzer fabricated on a silicon wafer. IEEE Trans. Electron. Dev. 26, 1880-1886 (1979).
17. Golden, A. P. & Tien, J. Fabrication of microfluidic hydrogels using molded gelatin as a sacrificial element. Lab Chip 7, 720-725 (2007).
18. Sung, K. E. et al. Control of 3-dimensional collagen matrix polymerization for reproducible human mammary fibroblast cell culture in microfluidic devices. Biomaterials 30, 4833-4841 (2009).
19. Vickerman, V., Blundo, J., Chung, S. & Kamm, R Design, fabrication and implementation of a novel multi-parameter control microfluidic platform for three-dimensional cell culture and real-time imaging. Lab Chip 8, 1468-1477(2008).
20. Sanchez-Freire, V., Ebert, A. D., Kalisky, T., Quake, S. R. & Wu, J. C. Microfluidic single-cell real-time PCR for comparative analysis of gene expression patterns. Nat. Protoc. 7, 829-838 (2012).
21. Kang, G., Ward, T. M., Bockhorn, J., Pegram, M. D. & Herr A. E. HER2 protein isoform heterogeneity investigated by single-cell western blotting. Cancer Res. 76, https://doi.org/10.1158/1538-7445.AM2016-352(2016).
22. Koh, A. et al. A soft, wearable microfluidic device for the capture, storage, and colorimetric sensing of sweat. Sci. Transl. Med. 8, 366ra165 (2016).
23. Huh, D. et al. Microfabrication of human organs-on-chips. Nat. Protoc. 8, 2135-2157 (2013).24. Bhatia, S. N. & Ingber, D. E. Microfluidic organs-on-chips. Nat. Biotechnol. 32, 760-772 (2014).
25. Hansen, W. A. et al. Supravital dithizone staining in the isolation of human and rat pancreatic islets. Diabetes Res. 10, 53-57 (1989).
26. Moon, C. et al. Compartmentalized accumulation of cAMP near complexes of multidrug resistance protein 4 (MRP4) and cystic fibrosis transmembrane conductance regulator (CFTR) contributes to drug-induced diarrhea. J. Biol. Chem. 290, 11246-11257 (2015).
27. Rock, J. R. et al. Basal cells as stem cells of the mouse trachea and human airway epithelium. Proc. Natl. Acad. Sci. USA 106, 12771 (2009).
28. Broutier, L. et al. Culture and establishment of self-renewing human and mouse adult liver and pancreas 3D organoids and their genetic manipulation. Nat. Protoc. 11, 1724-1743 (2016).
29. Mahe, M. M., Sundaram, N., Watson, C. L., Shroyer, N. F. & Helmrath, M. A. Establishment of human epithelial enteroids and colonoids from whole tissue and biopsy. J. Vis. Exp. https://doi.org/10.3791/52483(2015).
30. Ingber, D. E. Developmentally inspired human “organs on chips”. Development 145, https://doi.org/10.1242/dev.156125(2018).
31. Maleth, J. et al. Alcohol disrupts levels and function of the cystic fibrosis transmembrane conductance regulator to promote development of pancreatitis. Gastroenterology 148, 427-439 e416 (2015).
32. Toepke, M. W. & Beebe, D. J. PDMS absorption of small molecules and consequences in microfluidic applications. Lab Chip 6, 1484-1486 (2006).
33. van Meer, B. J. et al Small molecule absorption by PDMS in the context of drug response bioassays. Biochem. Biophys. Res. Commun. 482, 323-328(2017).
34. Tan, S. H., Nguyen, N. T., Chua, Y. C. & Kang, T. G. Oxygen plasma treatment for reducing hydrophobicity of a sealed polydimethylsiloxane microchannel. Biomicrofluidics 4, 32204 (2010).
35. Bodas, D. & Khan-Malek, C. Hydrophilization and hydrophobic recovery of PDMS by oxygen plasma and chemical treatment—An SEM investigation. Sens.

Actuators B123, 368-373 (2007).
36. Recek, N. et al. Adsorption of proteins and cell adhesion to plasma treated polymer substrates. Int. J. Polym. Mater. Polym. Biomater. 63, 685-691(2014).
37. Muck, A. et al. Fabrication of oly(methyl methacrylate) microfluidic chips by atmospheric molding. Anal. Chem. 76, 2290-2297 (2004).
38. Do, J. et al. Development of functional lab-on-a-chip on polymer for point-of-care testing of metabolic parameters. Lab Chip 8, 2113-2120 (2008).
39. Domansky, K. et al. SEBS elastomers for fabrication of microfluidic devices with reduced drug absorption by injection molding and extrusion. Microfluid. Nanofluidics 21, 107 (2017).
40. Roman, G. T., Hlaus, T., Bass, K. J., Seelhammer, T. G. & Culbertson, C. T.Sol-gel modified poly(dimethylsiloxane) microfluidic devices with high electroosmotic mobilities and hydrophilic channel wall characteristics. Anal. Chem. 77, 1414-1422 (2005).
41. Ostuni, E., Chen, C. S., Ingber, D. E. & Whitesides, G. M. Selective deposition of proteins and cells in arrays of microwells. Langmuir 17, 2828-2834 (2001).
42. Kelly, 0. G. et al. Cell-surface markers for the isolation of pancreatic cell types derived from human embryonic stem cells. Nat. Biotechnol. 29, 750-756(2011).
43. Watanabe, K. et al. A ROCK inhibitor permits survival of dissociated human embryonic stem cells. Nat. Biotechnol. 25, 681-686 (2007).
44. Schneider, F., Draheirn, J., Kamberger, R. & Wallrabe, U. Process and material properties of polydimethylsiloxane (PDMS) for optical MEMS. Sens. Actuators A151, 95-99 (2009).
45. Vannoort, R. & Bayston, R. Mechanical-properties of antibacterial silicone-rubber for hydrocephalus shunts. J. Biomed. Mater. Res. 13, 623-630 (1979).
46. Li, C. et al. Spatiotemporal coupling of cAMP transporter to CFTR chloride channel function in the gut epithelia. Cell 131, 940-951 (2007).
47. Arora, K. et al. Stabilizing rescued surface-localized delta f508 CFTR by potentiation of its interaction with Na(+)/H(+) exchanger regulatory factor 1. Biochemistry 53, 4169-4179 (2014)

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. All accessioned information (e.g., as identified by PUBMED, PUBCHEM, NCBI, UNIPROT, or EBI accession numbers) and publications in their entireties are incorporated into this disclosure by reference in order to more fully describe the state of the art as known to those skilled therein as of the date of this disclosure. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

1. A microfluidic device, comprising:
a first surface at least partially defining a first chamber;
a plurality of pancreatic ductal epithelial cells (PDECs) received within said first chamber;
a second surface at least partially defining a second chamber;
a plurality of pancreatic islets received within said second chamber; and
a permeable membrane fluidly connecting said first and second chambers such that said plurality of PDECs are configured to communicate with said plurality of pancreatic islets to mimic in situ pancreatic cell function.

2. The device of claim 1, wherein said PDECs and pancreatic islets are derived from an individual, wherein said individual may have a disease state selected from one or more of Acute recurrent pancreatitis (ARP) or chronic pancreatitis (CP), and cystic fibrosis (CF).

3. The microfluidic device of claim 1, wherein said first chamber further includes a first cell culture media positioned therein, and wherein said second chamber further includes a second cell culture media positioned therein.

4. The microfluidic device of claim 3, wherein said first cell culture media and said second cell culture media comprise insulin.

5. The microfluidic device of claim 1, wherein each of said plurality of PDECs is in a monolayer.

6. The microfluidic device of claim 1, wherein said plurality of PDECs is configured to express a cystic fibrosis transmembrane conductance regulator (CFTR) protein.

7. The microfluidic device of claim 1, wherein said plurality of islets is configured to secrete insulin.

8. The microfluidic device of claim 1, wherein said permeable membrane comprises a plurality of openings extending between and fluidly connecting said first and second chambers, and wherein each of said plurality of openings has of a width of from about 5 μm to about 25 μm, or about 10 μm.

9. The microfluidic device of claim 1, wherein said first surface is in contact with said plurality of PDECs, wherein said second surface is in contact with said plurality of pancreatic islets, and wherein at least one of said first surface or said second surface at least partially includes a hydrophilic surface.

10. The microfluidic device of claim 9, wherein said hydrophilic surface is selected from poly methyl methacrylate, acrylonitrile butadiene styrene copolymer, cyclic olefin copolymer, styrene ethylene butylene styrene, collagen, or combinations thereof.

11. The microfluidic device of claim 1, wherein said first surface is in contact with said plurality of PDECs, wherein said second surface is in contact with said plurality of pancreatic islets, and wherein at least one of said first surface or said second surface has a sol-gel-modified PDMS or a collagen-coated-PDMS received thereon.

12. The microfluidic device of claim 1, wherein said first chamber includes a first branch channel and a second branch channel, wherein each of said first and second branch channels extend in a common channel plane and intersect at a first predetermined angle.

13. The microfluidic device of claim 1, wherein said first branch channel further includes a first pair of side edges extending in the common channel plane and defines a first width therebetween, wherein said second branch channel further includes a second pair of side edges extending in the common channel plane and defining a second width therebetween, and wherein the second width is narrower than the first width.

14. A method of measuring cystic fibrosis transmembrane conductance regulator (CFTR) protein function in an individual, comprising
a. obtaining pancreatic ductal epithelial cells (PDECs) and pancreatic islets from said individual;
b. culturing said PDECs and pancreatic islets in the device of claim 1, wherein patient-derived pancreatic ductal epithelial cells (PDECs) are co-cultured in a first chamber, and patient-derived pancreatic islet cells are cultured in second chamber;
c. assaying the function of said CFTRs in a pancreatic ductal monolayer; and
d. measuring insulin secretion of said pancreatic islets.

15. The method of claim 14 further comprising measuring one or more of fluid secretion from said PDECs in response to forskolin and measuring insulin secretion of said pancreatic islets in response to glucose.

16. The method of claim 14 wherein said individual has Cystic Fibrosis (CF)-related diabetes (CFRD).

17. The method of claim 14, wherein said first and/or second chamber are contacted with alcohol to determine one or both of CFTR function and endocrine function in response to said alcohol.

18. The method of claim 14, wherein said method is used to determine function of a CFTR mutation type, wherein said PDECs are known to contain said CFTR mutation type, and wherein function of one or both of said PDECs and/or pancreatic islets are correlated with said CRTR mutation type.

19. The method of claim 14, further comprising
a. contacting said first or second chamber with an agent suspected of improving glucose abnormalities; and
b. measuring a glucose response in said pancreatic islets in response to said contact.

20. A method of assaying a potential treatment for one or more of Acute Recurrent Pancreatitis (ARP) or Chronic Pancreatitis (CP), and Cystic Fibrosis (CF), and Cystic Fibrosis (CF)-related diabetes (CFRD), comprising
a. contacting a potential therapeutic agent with one or both of said first and said second chambers of the device of claim 1; and
b. detecting a desired output.

21. The method of claim 20, wherein said desired output is selected from one or both of fluid secretion from PDECs and insulin secretion from said pancreatic islets.

22. A method of making a pancreatic ductal epithelial cells (PDECs) monolayer, comprising digesting pancreatic duct tissue obtained from said individual, isolating PDECs from said digested pancreatic duct tissue, embedding said isolated PDECs in a matrix, and incubating with media until one or both of an organoid and a monolayer is formed.

23. The method of claim 22, wherein said matrix is disrupted mechanically, in the absence of trypsin, prior to said incubation with media to form said monolayer.

24. The method of claim 22, wherein said monolayer is a polarized monolayer.

25. The method of claim 22, wherein said pancreatic ductal epithelial cells express CFTR.
